(19) United States
(12) Patent Application Publication
(10) Pub. No.: US 2024/0228418 A1
(43) Pub. Date: Jul. 11, 2024

(54) INTEGRATION FOR PROCESSING EFFLUENT OF OXIDATIVE DEHYDROGENATION (ODH) REACTOR

(71) Applicant: NOVA CHEMICALS (INTERNATIONAL) S.A., Fribourg (CH)

(72) Inventors: Mohammad KESHTKAR, Calgary (CA); David GENT, Red Deer (CA)

(21) Appl. No.: 18/288,751

(22) PCT Filed: Apr. 26, 2022
(86) PCT No.: PCT/IB2022/053872

§ 371 Date: Oct. 27, 2023

Related U.S. Application Data
(60) Provisional application No. 63/181,102, filed on Apr. 28, 2021.

(51) Int. Cl. C07C 51/215; B01D 3/14; B01D 11/04; B01J 19/00; C07C 5/48; C07C 51/48

(52) U.S. Cl. CPC C07C 51/215; B01D 3/143; B01D 11/0492; B01J 19/0013; C07C 5/48; C07C 51/48; B01J 2219/00103

(57) ABSTRACT

A system and method for producing ethylene, including dehydrogenating ethane to ethylene via an ODH catalyst in an ODH reactor, discharging an effluent from the ODH reactor, heating feed including ethane to the ODH reactor with the effluent, recovering acetic acid from the effluent as acetic acid product, and forwarding a process gas including ethylene from the effluent for further processing to give ethylene product. The technique involves energy integration including with respect to the processing of the effluent. Water may be recovered from the effluent as recycle water for addition of the recycle water to the feed.

### CLAIM OF PRIORITY

This application claims priority to U.S. Provisional Application No. 63/181,102 filed on Apr. 28, 2021, the entire contents of which are hereby incorporated by reference.

### TECHNICAL FIELD

This disclosure relates to oxidative dehydrogenation (ODH) of ethane to produce ethylene.

### BACKGROUND ART

Catalytic oxidative dehydrogenation of alkanes into corresponding alkenes is an alternative to steam cracking. In contrast to steam cracking, oxidative dehydrogenation (ODH) may operate at lower temperature and generally does not produce coke. For ethylene production, ODH may provide a greater yield for ethylene than does steam cracking. ODH may be performed in a reactor vessel having a catalyst for the conversion of an alkane to a corresponding alkene. Acetic acid may be generated in the conversion of the lower alkanes (e.g., ethane) into the corresponding alkenes (e.g., ethylene).

Carbon dioxide is the primary greenhouse gas emitted through human activities. Carbon dioxide (CO₂) may be generated in various industrial and chemical plant facilities, including ODH facilities. At such facilities, more efficient utilization of energy may reduce CO₂ emissions at the facility and therefore decrease the CO₂ footprint of the facility.

### SUMMARY OF INVENTION

An aspect relates to a method of producing ethylene, including dehydrogenating ethane to ethylene via an oxidative dehydrogenation (ODH) catalyst in the presence of oxygen in an ODH reactor, thereby forming acetic acid in the ODH reactor, and discharging an effluent including at least ethylene, acetic acid, and water from the ODH reactor through a steam-generation heat exchanger to generate steam with heat from the effluent, thereby cooling the effluent. The method includes flowing the effluent from the steam-generation heat exchanger through a feed heat exchanger to heat a feed having ethane for the ODH reactor with the effluent, thereby cooling the effluent. The method includes recovering acetic acid from the effluent as acetic acid product and forwarding a process gas having ethylene from the effluent for further processing to give ethylene product.

Another aspect relates to a method of producing ethylene, including dehydrogenating ethane to ethylene via an ODH catalyst in the presence of oxygen in an ODH reactor, thereby forming acetic acid in the ODH reactor, and discharging an effluent including ethylene, acetic acid, water, carbon monoxide, carbon dioxide, and unreacted ethane from the ODH reactor through a steam-generation heat exchanger to generate steam, wherein the steam-generation heat exchanger transfers heat from the effluent to water to generate the steam, thereby cooling the effluent. The method includes flowing the effluent from the steam-generation heat exchanger through a feed heat exchanger to heat a feed for the ODH reactor with the effluent, wherein the feed heat exchanger transfers heat from the effluent to the feed, thereby cooling the effluent. The method includes cooling the effluent downstream of the feed heat exchanger, thereby condensing water in the effluent. The method includes forwarding process gas having ethylene from the effluent to a process gas compressor for further processing to give ethylene product.

Yet another aspect relates to a method of producing ethylene, including dehydrogenating ethane to ethylene via an ODH catalyst in the presence of oxygen in an ODH reactor, thereby forming acetic acid in the ODH reactor, and discharging an effluent including ethylene, acetic acid, water, carbon monoxide, carbon dioxide, and unreacted ethane from the ODH reactor through a steam-generation heat exchanger to generate steam and through a feed heat exchanger to heat a feed including ethane for the ODH reactor. The method includes separating the effluent in a vessel into gas and raw acetic acid, wherein the gas includes ethylene, water, acetic acid, ethane, carbon dioxide, and carbon monoxide, and wherein the raw acetic acid includes acetic acid and water. The method includes removing acetic acid and water from the gas to give process gas including ethylene, ethane, carbon dioxide, and carbon monoxide and forwarding the process gas to a process gas compressor for further processing to give ethylene product, wherein the process gas includes less than 50 part per million by volume (ppmv) of acetic acid (and less than 5 mole percent of water in some implementations). The method includes discharging the raw acetic acid from a bottom portion of the vessel to an acetic acid unit (having an extractor column) to recover acetic acid product from the raw acetic acid.

Yet another aspect relates to an ethylene production system including an ODH reactor having an ODH catalyst to dehydrogenate ethane to ethylene and generate acetic acid, a steam-generation heat exchanger to receive an effluent from the ODH reactor to generate steam with heat from the effluent, a feed heat exchanger to receive the effluent from the steam-generation heat exchanger to heat a feed including at least ethane for the ODH reactor with the effluent, and a vessel to separate the effluent into gas and raw acetic acid, wherein the gas includes ethylene, water, acetic acid, ethane, carbon dioxide, and carbon monoxide, and wherein the raw acetic acid includes acetic acid and water. The ethylene production system includes an acetic acid unit to process the raw acetic acid to give acetic acid product, wherein the acetic acid unit includes an extractor column that is a liquid-liquid extraction column.

Yet another aspect relates to a method of producing ethylene, including dehydrogenating ethane to ethylene via an ODH catalyst in an ODH reactor, and discharging an effluent from the ODH reactor, the effluent including ethylene, water, acetic acid, carbon dioxide, carbon monoxide, and unreacted ethane. The method includes condensing acetic acid and water in the effluent to separate the effluent into raw acetic acid and gas, the raw acetic acid including the condensed acetic acid and the condensed water, wherein the gas includes ethylene, carbon dioxide, carbon monoxide, and unreacted ethane. The method includes processing the raw acetic acid to give acetic acid product and processing the gas to give process gas including ethylene product. The method includes recovering water from the effluent as recycle water, adding the recycle water to the feed including ethane to the ODH reactor, heating the feed with the effluent, and adding oxygen to the feed.

The details of one or more implementations are set forth in the accompanying drawings and the description below. Other features and advantages will be apparent from the description and drawings, and from the claims.

### BRIEF DESCRIPTION OF DRAWINGS

FIG. 1 is a process flow diagram (PFD) of an ethylene production system according to Option 1 (base case).

FIG. 2 is a PFD of an ethylene production system according to Option 2.

FIG. 3 is a PFD of an ethylene production system according to Option 3.

FIG. 4 is a PFD of an ethylene production system according to Option 4.

FIG. 5 is a PFD of an ethylene production system according to Option 5.

FIG. 6 is a PFD of an ethylene production system according to Option 6.

FIG. 7 is a PFD of an ethylene production system according to Option 8.

FIG. 8 is a PFD of an ethylene production system according to Option 9.

FIG. 9 is a PFD of an ethylene production system according to Option 10.

FIG. 10 is a PFD of an ethylene production system according to a variation of Option 10.

FIG. 11 is a PFD of an ethylene production system according to Option 11.

FIG. 12 is a PFD of an ethylene production system according to a variation of Option 11.

FIG. 13 is a PFD of an ethylene production system according to Option 12.

FIG. 14 is a PFD of an ethylene production system according to Option 13.

FIG. 15 is a PFD of an ethylene production system according to Option 14.

FIG. 16 is a PFD of an ethylene production system according to Option 15.

FIG. 17 is a PFD of an ethylene production system according to Option 16.

FIG. 18 is a PFD of an ethylene production system according to Option 17.

FIG. 19 is a PFD of an ethylene production system according to Option 18.

FIG. 20 is a PFD of an ethylene production system according to Option 19.

FIG. 21 is a PFD of an ethylene production system according to Option 20.

FIG. 22 is a PFD of an ethylene production system according to Option 21.

FIG. 23 is a PFD of an ethylene production system according to Option 22.

FIG. 24 is a block flow diagram of a method of producing ethylene.

Like reference numbers and designations in the various drawings indicate like elements.

### DESCRIPTION OF EMBODIMENTS

Some aspects of disclosure are directed to dehydrogenating ethane to ethylene via an oxidative dehydrogenation (ODH) catalyst in the presence of oxygen in an ODH reactor. Acetic acid is also formed in the ODH reactor. The technique may include discharging an effluent including ethylene, acetic acid, and water from the ODH reactor through a steam-generation heat exchanger to generate steam and also through a feed heat exchanger (cross-exchanger) to heat a feed including ethane for the ODH reactor. Raw acetic acid can be separated from the effluent. The raw acetic acid may be the majority of the water and acetic acid in the effluent that is condensed to promote separation from the effluent. The raw acetic acid may be processed in an acetic acid unit to give acetic acid product. Gases including ethylene, unreacted ethane, carbon dioxide, carbon monoxide, uncondensed acetic acid, and uncondensed water can be separated from the effluent and scrubbed to remove acetic acid and water to give process gas. In implementations, the process gas may be forwarded to a process gas compressor for further processing to give ethylene product.

Energy integration (e.g., energy recovery from reactor effluent) and increasing the overall energy efficiency of the ODH reactor system including downstream processing of reactor effluent may be beneficial to decrease operating expense and emissions of greenhouse gases, such as carbon dioxide. Energy integration of reactor effluent cooling, acetic acid recovery, and reactor feed saturation is disclosed. The described energy integration can reduce steam consumption, power demand, and cooling-water demand while advantageously concentrating raw acetic acid to the acetic acid unit. Such may generally result not only in overall lower operating expense of the ODH reactor plant but also in lower capital expense for at least the acetic acid unit, cooling water (CW) system, and steam system. The integration for the ODH reactor system can also include recirculation of water from the reactor effluent to the reactor feed dilution. This water recovered in the processing of the effluent can be labeled as recycle water.

Options of the energy integration of reactor effluent cooling, acetic acid recovery, and reactor feed saturation are given. The aforementioned recycle water is considered. The example of Option 1 presented below may be a base case. Other options presented may be generally compared to Option 1 as a baseline case. However, the present techniques are not limited to the various options as tabulated or characterized. Instead, the various options as configured including Options 1-22 are given as examples.

The ODH reaction to dehydrogenate feed ethane to product ethylene and generate byproduct acetic acid may occur at a temperature, for example, between 300-450° C. with low-temperature ODH catalyst (e.g., MoVNbTeOx as discussed below) to produce ethylene with high selectivity. To stay outside of flammability envelope of ethane-oxygen mixture in the feed and ODH reactor, a diluent is employed. Vaporized water or steam can be used as the diluent. Based on pressure and temperature of mixed feed including ethane, oxygen, and water to the ODH reactor, the target oxygen concentration can differ. Several process configuration schemes (e.g., including an ethane saturator tower) may be implemented to mix water as diluent with ethane and oxygen. Heat-integrations options including different cooling schemes for the ODH reactor effluent are compared.

Two major heat demands in the ODH reaction process to produce ethylene may be: (1) feed saturation to dilute the mixed feed; and (2) solvent recovery tower in the acetic acid (AA) unit that gives the AA product stream. Two main cooling demands for this process may be: (1) reactor effluent cooling; and (2) condensing the overhead stream from the solvent recovery tower of the AA unit.

Embodiments may be directed to process integration to cool down the reactor effluent from an ODH reactor. In presented options, reactor effluent discharging from the reactor can be initially used to generate or superheat (very) high pressure steam and then the effluent is cross-exchanged against reactor feed.

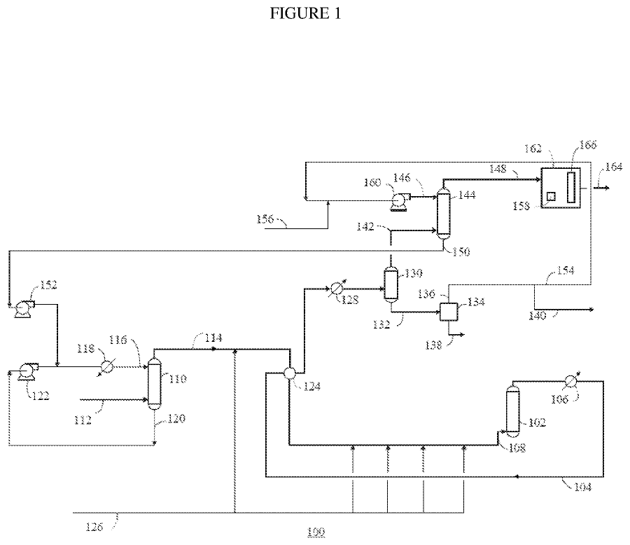

FIG. 1 is an ethylene production system 100. FIG. 1 as depicted may be characterized as Option 1 for comparison to subsequent figures. The ethylene production system 100 includes an ODH reactor 102 vessel that has an ODH catalyst to dehydrogenate ethane to ethylene. The operating temperature of the reactor may be, for example, in the range of 300° C. to 450° C. The ODH reaction may typically be exothermic. The ODH reactor 102 system may utilize a heat-transfer fluid for controlling temperature of the ODH reactor 102. The heat-transfer fluid may be employed to remove heat from (or add heat to) the ODH reactor 102. The heat transfer fluid can be, for example, steam, water (including pressurized or supercritical water), oil, or molten salt, and so forth. The ODH reactor 102 may be, for example, a fixed-bed reactor (operating with a fixed bed of ODH catalyst) or a fluidized-bed reactor (operating with a fluidized bed of catalyst), or another reactor type.

For the ODH reactor as a fixed-bed reactor, reactants may be introduced into the reactor at one end and flow past an immobilized catalyst. Products are formed and an effluent having the products may discharge at the other end of the reactor. The fixed-bed reactor may have one or more tubes (e.g., metal tubes, ceramic tubes, etc.) each having a bed of catalyst and for flow of reactants. For the ODH reactor 102, the flowing reactants may be at least ethane and oxygen. The tubes may include, for example, a steel mesh. Moreover, a heat-transfer jacket adjacent the tube(s) or an external heat exchanger (e.g., feed heat exchanger or recirculation heat exchanger) may provide for temperature control of the reactor. The aforementioned heat transfer fluid may flow through the jacket or external heat exchanger.

The ODH reactor as a fluidized bed reactor can be (1) a non-circulating fluidized bed, (2) a circulating fluidized bed with regenerator, or (3) a circulating fluidized bed without regenerator. In implementations, a fluidized bed reactor may have a support for the ODH catalyst. The support may be a porous structure or distributor plate and disposed in a bottom portion of the reactor. Reactants may flow upward through the support at a velocity to fluidize the bed of ODH catalyst. The reactants (e.g., ethane, oxygen, etc. for the ODH reactor 102) are converted to products (e.g., ethylene and acetic acid in the ODH reactor 102) upon contact with the fluidized catalyst. An effluent having products may discharge from an upper portion of the reactor. A cooling jacket may facilitate temperature control of the reactor. The fluidized bed reactor may have heat-transfer tube, a jacket, or an external heat exchanger (e.g., feed heat exchanger or recirculation loop heat exchanger) to facilitate temperature control of the reactor. The aforementioned heat transfer fluid may flow through the reactor tube, jacket, or external heat exchanger.

As indicated, the ODH catalyst may be operated as a fixed bed or fluidized bed. An ODH catalyst that can facilitate an ODH reaction that dehydrogenates ethane to ethylene and forms acetic acid as a byproduct may be applicable to the present techniques. Mixed metal oxide catalysts are particularly well suited for ethane ODH and for use in the methods and ethylene production systems described herein. A low-temperature ODH catalyst may be beneficial. One example of an ODH catalyst that may be utilized in the ODH reactor is a low-temperature ODH mixed metal oxide catalyst that includes molybdenum, vanadium, tellurium, niobium, and oxygen, wherein the molar ratio of molybdenum to vanadium is from 1:0.12 to 1:0.49, the molar ratio of molybdenum to tellurium is from 1:0.01 to 1:0.30, the molar ratio of molybdenum to niobium is from 1:0.01 to 1:0.30, and oxygen is present at least in an amount to satisfy the valency of any present metal elements. The molar ratios of molybdenum, vanadium, tellurium, niobium can be determined by inductively coupled plasma mass spectrometry (ICP-MS). The catalyst may be low temperature in providing for the ODH reaction at less than 450° C., less than 425° C., or less than 400° C. Another example of a mixed metal oxide catalyst includes molybdenum, vanadium, tellurium, and tantalum.

In the ODH reaction that dehydrogenates the ethane, a byproduct formed may be acetic acid. Also formed in the ODH reaction may include water, carbon dioxide, and carbon monoxide. Thus, the effluent 104 discharged from the ODH reactor 102 vessel may include ethylene, acetic acid, water, carbon dioxide, carbon monoxide, and unreacted ethane. The operating temperature of the ODH reactor 102 and the temperature of the effluent 104 as discharged may be, for example, in the range of 300° C. to 450° C.

The effluent 104 may be routed through a conduit to a steam-generation heat exchanger 106 to generate steam with heat from the effluent 104. The steam-generation heat exchanger 106 may be, for example, a shell-and-tube heat exchanger or a fin-type heat exchanger (e.g., with a finned-tube bundle), and so on. The effluent 104 may be cooled by at least 100° C. across the steam-generation heat exchanger 106.

Water may be heated in the steam-generation heat exchanger 106 with heat from the effluent 104 to flash the water into steam. The water may be, for example, boiler feedwater, demineralized water, or steam condensate, and the like. More than one steam-generation heat exchanger 106 may be employed in series and/or parallel. The steam generation system having the steam-generation heat exchanger 106 may include additional equipment, such as a vessel (e.g., flash vessel), a pump (e.g., boiler feedwater pump), etc. The steam generated may discharge into a steam header (or sub-header) conduit or through a conduit to a user, and so on. Higher pressure steam may generally be more valuable than lower pressure steam.

Higher pressure steam, such as greater than 600 pounds per square inch gauge (psig) or greater than 1500 psig, may typically be more valuable than lower pressure steam, such as less than 600 psig or less than 150 psig. The pressure of the steam generated via the steam-generation heat exchanger 106 may be a function of the temperature of the effluent 104 driven by the operating temperature (ODH reaction temperature) of the ODH reactor 102.

An ethane saturator tower 110 may provide ethane (e.g., water-saturated ethane that is ethane saturated in water) for the mixed feed 108 to the ODH reactor 102 vessel. The ethylene production system 100 may include the ethane saturator tower 110 vessel (e.g., column) to incorporate water vapor into ethane gas 112 and discharge saturated ethane 114 for the mixed feed 108.

In implementations, liquid water 116 may enter an upper portion of the ethane saturator tower 110 and flow downward through the ethane saturator tower 110. The ethane saturator tower 110 may have an inlet (e.g., nozzle) that is a flanged or screwed connection with the conduit conveying the incoming water 116. The ethane gas 112 may enter a lower portion of the ethane saturator tower 110 and flow upward through the ethane saturator tower 110. The ethane saturator tower 110 may have an inlet (e.g., nozzle) that is a flanged or screwed connection with the conduit conveying the incoming ethane gas 112. The ethane saturator tower 110 may have packing or trays to provide contact stages of the ethane gas 112 with the water 116 for mass transfer of water vapor into the ethane gas 112. The ethane saturator tower 110 may include random packing, structured packing, or trays, or any combinations thereof.

Liquid water 120 may discharge (e.g., as a bottoms stream) from a bottom portion of the ethane saturator tower 110 and be recirculated via a water recirculation pump 122 (e.g., centrifugal pump) as water feed to the ethane saturator tower 110. Thus, the ethane saturator tower 110 may have a water recirculation loop. The water may be heated in a circulation-water heater 118 (e.g., shell-and-tube heat exchanger) with a heating medium such as steam to give the liquid water 116 (as heated) that enters the ethane saturator tower 110. The saturated ethane 114 may discharge overhead from the ethane saturator tower 110 for feed to the ODH reactor 102. The term “saturated” ethane as used herein means that the ethane gas is saturated with water. The saturated ethane 114 generally includes water vapor but little or no liquid water.

The saturated ethane 114 may be routed through a feed heat exchanger 124 that heats the saturated ethane 114 as feed to the ODH reactor 102. The feed heat exchanger 124 may be, for example a shell-and-tube heat exchanger or a plate-fin heat exchanger. In implementations, the feed heat exchanger 124 may be a cross exchanger, as depicted, with the effluent 104 heating the saturated ethane 114. The effluent 104 may thus be cooled in the feed heat exchanger 124, e.g., typically by at least 100° C. In other implementations, the feed heat exchanger 124 may utilize steam instead of the effluent 104 as the heating medium.

Oxygen (02) gas 126 may added to the saturated ethane gas 112 upstream of the feed heat exchanger 124 or downstream of the feed heat exchanger 124, or both. The oxygen gas 126 may be added to the saturated ethane at a single addition point or at multiple addition points (e.g., 2-5 addition points). The illustrated embodiment depicts five addition points. A reason for multiple addition points may be to reduce the chance of forming a pocket of oxygen gas 126 in the flowing saturated ethane 114.

The oxygen gas 126 may be added to a conduit conveying the saturated ethane 114. In implementations, the conduit may include an in-line static mixer that is adjacent (downstream) of the addition point of the oxygen gas 126 into the saturated ethane 114. In implementations, the conduit conveying the oxygen gas 126 may tie-in to the conduit conveying the saturated ethane 114 via a pipe tee or similar pipe fitting. The mixed feed 108 to the ODH reactor 102 may include the saturated ethane gas 112 and the oxygen gas 126. As indicated, the water in the saturated ethane gas 112 may be a diluent.

The effluent 104 flows from the feed heat exchanger 124 through a cooler heat exchanger 128 to a flash drum 130. The flash drum 130 is a vessel, e.g., with a vertical orientation or horizontal orientation. In implementations, a level of liquid (e.g., raw acetic acid that may be primarily water) may be maintained in the flash drum 130 in operation.

The cooler heat exchanger 128 cools (removes heat from) the effluent 104. The cooling medium may be, for example, cooling tower water. The cooler heat exchanger 128 may be, for example, a shell-and-tube heat exchanger or plate-fin heat exchanger, or other type of heat exchanger. In implementations, the cooler heat exchanger 128 discharges the effluent 104 at a temperature, for example, in a range of 30° C. to 80° C. The cooler heat exchanger 128 may be a condenser in that water and acetic acid in the effluent 104 can condense in the cooler heat exchanger 128.

The operating pressure of the flash drum 130 may be a function of the backpressure of downstream processing of process gas (discussed below). The operating pressure of the flash drum 130 may be a function of the ODH reactor 102 discharge pressure of the effluent 104. The operating pressure of the flash drum 130 may be a function of the pressure drop associated with the flow of the effluent 104 from the ODH reactor 102 through the piping and heat exchangers to the flash drum 130 and to the downstream process gas compressor.

The temperature of the effluent 104 entering the flash drum 130 may be affected by the amount of cooling of the effluent 104 in the feed heat exchanger 124 and the cooler heat exchanger 128. The amount of water in the raw acetic acid 132 discharged as a bottoms stream from the flash drum 130 may be a function of the temperature of the effluent 104 that enters the flash drum 130. A lower temperature of the effluent 104 entering the flash drum 130 may give more water in the raw acetic acid 132. This may be so because more water will be condensed in the effluent 104 at lower temperatures. The raw acetic acid 132 may be primarily water. The terms “primarily” or “majority” as used herein mean greater than half (greater than 50 percent) including greater than 50 weight percent and greater than 50 volume percent.

An aspect of Option 1 is to cool the ODH reactor effluent 104 in the cooler heat exchanger 128 against cooling water (e.g., down to a temperature in a range of 30° C. to 80° C.) to condense a majority of the water and acetic acid in the ODH reactor effluent 104. Therefore, because a majority of water is condensed, the raw acetic acid 132 that discharges from the flash drum 130 in this embodiment may have a significant amount of water. Thus, the raw acetic acid 132 may have a low concentration of acetic acid, such as less than 1 weight percent (wt %). Depending on the embodiment and temperature of the effluent 104 entering the flash drum 130, the concentration of acetic acid in the raw acetic acid 132 can range, for example, from 0.3 wt % to 45 wt %.

The flash drum 130 discharges the raw acetic acid 132 from a bottom portion of the flash drum 130. The raw acetic acid 132 incudes liquid acetic acid and liquid water. The flash drum 130 may have outlet on the bottom portion of the flash drum 130 for the discharge of the raw acetic acid 132. The outlet may be a flanged nozzle or screwed nozzle that couples to a conduit for discharge of the raw acetic acid 132 from the flash drum 130 into the conduit. The flash drum 130 may discharge the raw acetic acid 132 through the conduit to an acetic acid unit 134, e.g., such as to an extractor column in the acetic acid unit 134.

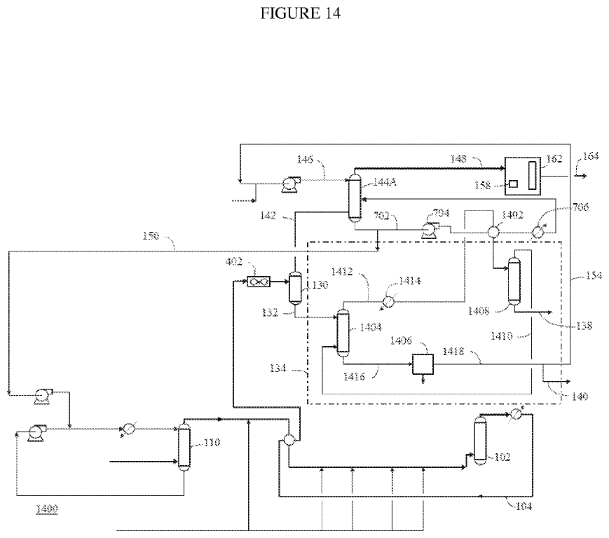

The raw acetic acid 132 may be processed in the acetic acid unit 134 to remove water 136 from the raw acetic acid 132 to give acetic acid product 138 that is a coproduct of the ethylene production. The acetic acid product 138 may be, for example, have at least 99 wt % acetic acid. At least a portion of the water 136 removed may be recovered as water product 140. As discussed below (e.g., with respect to FIG. 14), the acetic acid unit 134 may include an extractor column (vessel) for injection of solvent to remove acetic acid, a water stripper tower (vessel) to process raffinate from the extractor column to recover water, and a solvent recovery column (vessel) to remove the solvent from the acetic acid discharged from the extractor column to give the acetic acid product 138.

The flash drum 130 may discharge gas 142 overhead from a top portion of the flash drum 130. The gas 142 may include water vapor, residual acetic-acid vapor, and gases such as ethylene, carbon dioxide, carbon monoxide, unreacted ethane, and other gases. The other gases may include, for example, relatively small amounts of methane or propane that entered the system 100 with the ethane gas 112 (e.g., from pipeline). The flash drum 130 may include an outlet on a top portion of the flash drum 130 for discharge of the gas 142. The outlet may be a nozzle with a flange or screwed fitting to couple to a discharge conduit for discharge of the gas 142. The gas 142 may flow through the discharge conduit to an acetic acid scrubber 144, which is a vessel such as a tower or column.

A purpose of the acetic acid scrubber 144 may be to scrub (remove) acetic acid and water from the gas 142. The acetic acid and water removed may generally be the remainder of the acetic acid and water from the effluent 104 that was condensed to give the raw acetic acid 132. In some implementations, this removal of the acetic acid from the gas 142 giving ppm levels (e.g., <50 ppm) of acetic acid in the process gas 148 may reduce the metallurgical cost of downstream process equipment (e.g., process gas compressor 158, etc.). In implementations, the process gas 148 may include trace amounts of acetic acid, such as less than 50 part per million by volume (ppmv) of acetic acid. The process gas 148 may also include a small amount of water, such as less than 5 mole percent (mol %) of water.

The scrubbing liquid may be scrubbing water 146 that enters an upper portion of the acetic acid scrubber 144 and flows downward through the acetic acid scrubber 144. The scrubber 144 may have an inlet, such as a nozzle, for receiving the scrubbing water 146. The nozzle may be, for example, a flanged or screwed connection coupled with the inlet conduit conveying the incoming scrubbing water 146. The gas 142 from the flash drum 130 may enter a lower portion of the scrubber 144 vessel and flow upward through the scrubber 144 in a countercurrent flow with respect to the scrubbing water 146. The scrubber 144 may have an inlet (e.g., nozzle) that is a flanged or a screwed connection with the inlet conduit conveying the incoming gas 142. The acetic acid scrubber 144 may have packing or trays to provide contact stages of the gas 142 with the scrubbing water 146 for mass transfer of water vapor and acetic acid vapor from the gas 142 into the scrubbing water 146. The scrubber 144 may include random packing, ordered packing, or trays, or any combinations thereof.

The acetic acid scrubber 144 may discharge process gas 148 (e.g., overhead stream) for downstream processing to recover ethylene product. The process gas 148 may include ethylene, ethane, carbon dioxide, carbon monoxide, propane, and methane. The mole percent (mol %) of ethylene in the process gas 148 may be, for example, in the range of 10 mol % to 90 mol %. The process gas 148 is generally the gas 142 minus the acetic acid vapor and water vapor removed from the gas 142 in the scrubber 144. The process gas 148 may discharge through an outlet nozzle on a top portion of the scrubber 142, and in which the nozzle is coupled to a discharge conduit.

The scrubbing water 146 having the acetic acid vapor and water vapor removed from the gas 142 may discharge as a bottoms stream (through an outlet nozzle on a bottom portion of the scrubber 144) as recycle water 150 to the ethane saturator tower 110. The recycle water 150 may flow through a conduit to the ethane saturator tower 110. A recycle water pump 152 may be disposed along the conduit to provide motive force for flow of the recycle water 150. The recycle water 150 may be combined with the bottoms liquid water 120 from the ethane saturator tower 110, and flow through the circulation water heater 118 as the liquid water 116 feed to the ethane saturator tower 110.

The scrubbing water 146 fed to the acetic acid scrubber 144 may include, for example, liquid water 154 from the acetic acid unit 134 and water condensate 156 from a downstream process gas compressor (PGC) 158. A pump 160 may provide motive force for flow of the scrubbing water 146 to the acetic acid scrubber 144.

The process gas 148 discharged from the acetic acid scrubber 144 may be processed by downstream equipment 162 to remove ethylene from the process gas 148 as product ethylene 164. The downstream equipment 162 may include the aforementioned PGC 158 (e.g., mechanical compressor) that increases the pressure of the process gas 148. The compressed process gas may be processed to remove light components, such as carbon monoxide and methane. The downstream equipment 162 may include a C2 splitter 166 that separates ethylene from ethane. The C2 splitter 166 may be a vessel that is a distillation column having distillation trays.

In an embodiment, the ethylene production system 100 forwards the process gas 142 to the downstream equipment 162 but does not include the downstream equipment 162. Instead, the product of the ethylene production system 100 is the process gas 148 having ethylene. In another embodiment, the ethylene production system 100 includes the PGC compressor 158 that discharges the process gas 148 as product. In yet another embodiment, the ethylene production system 100 includes the downstream process equipment 162. The discussion or analysis of energy among the Options 1-22 consider the PGC 158 but typically not the remaining equipment in the downstream equipment 162.

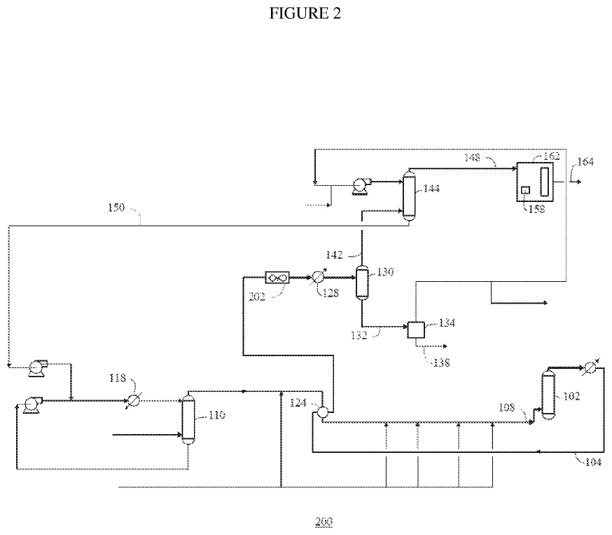

FIG. 2 is an ethylene production system 200 that is the same or similar as the ethylene production system 100 of FIG. 1 but with the addition of an air cooler 202. FIG. 2 may be characterized as Option 2. For a description of equipment and reference numerals depicted in FIG. 2, see the discussion of FIG. 1. The air cooler 202 cools (removes heat from) the ODH reactor effluent 104 with ambient air as the cooling medium or heat transfer fluid. The air cooler 202 may be operationally disposed between the feed heat exchanger 124 and the cooler heat exchanger 128. The air cooler 202 is a heat exchanger that may be fan heat exchanger including one or more fans. The fan heat exchanger may have fins. The air cooler 202 may be a fin-fan heat exchanger. The air cooler 202 heat exchanger may have one or more fans with a finned-tube bundle, and the like.

The air cooler 202 may discharge the effluent 104 at a temperature in a range of 80° C. to 130° C., or in range of 80° C. to 100° C. As with Option 1, the cooler heat exchanger 128 may discharge the effluent 104 at a temperature, for example, in a range of 30° C. to 80° C. Thus, a majority of the water and acetic acid in the effluent 104 is condensed. Therefore, as with Option 1, the raw acetic acid 132 may have a low concentration of acetic acid, such as less than 1 wt %. Such may be labeled as low acetic-acid in the raw acetic acid.

However, the removal of heat from the effluent 104 to cool the effluent 104 down to a temperature in a range of temperature 30° C. to 80° C. is shared by the air cooler 202 and the cooler heat exchanger 128. Therefore, the cooling medium (e.g., cooling tower water) demand by the cooler heat exchanger 128 is reduced as compared to Option 1. Accordingly, in implementations, the size of the cooling tower and its operating cost may be beneficially reduced. However, the addition of the extra equipment (air cooler 202) may increase pressure drop of the effluent 104, which could translate to more power consumption at the PGC 158.

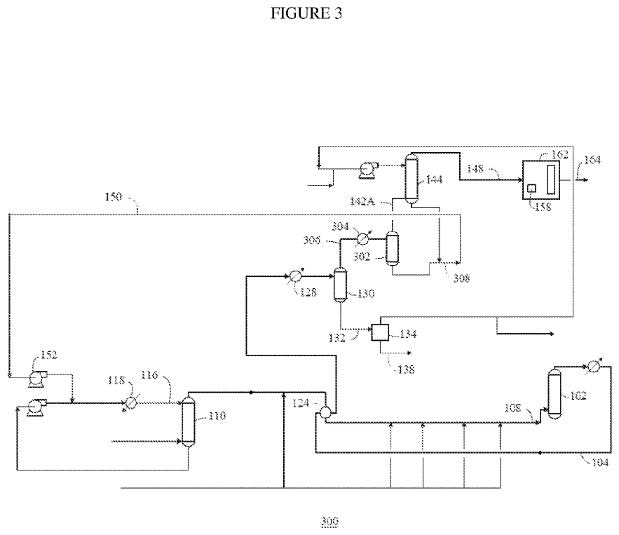

FIG. 3 is an ethylene production system 300 that is the same or similar as the ethylene production system 100 of FIG. 1 but with the addition of the second flash drum 302 (vessel) and the second cooler heat exchanger 304. The second cooler heat exchanger 304 may use water (e.g., cooling tower water) as the cooling medium. FIG. 3 may be characterized as Option 3. For a description of equipment and reference numerals depicted in FIG. 3, see also the discussion of FIG. 1.

The first cooler heat exchanger 128 may cool the effluent 104, for example, to a temperature in a range of 30° C. to 120° C., or in a range of 80° C. to 120° C.

In Options 1 and 2 previously discussed, the cooler heat exchanger 128 may cool the effluent 104, for example, to in the range of 30° C. to 80° C., and thus may condense a majority of the acetic acid in the effluent 104 and a majority of the water in the effluent 104. Therefore, in Options 1-2, the raw acetic acid 132 that discharges from the flash drum 130 may have a low concentration (e.g., less than 1 wt %) of acetic acid and may be labeled as low acetic-acid concentration with a relatively high overall flow (more load) to acetic acid unit 134.

In contrast, in Option 3, the first cooler heat exchanger 128 may cool the effluent 104, for example, to a temperature in the aforementioned range of 80° C. to 120° C. Thus, the first cooler heat exchanger 128 may condense a majority of the acetic acid in the effluent 104 but less than a majority of the water in the effluent 104. Therefore, the raw acetic acid 132 that discharges from the flash drum 130 may have a higher concentration (e.g., at least 1 wt %) of acetic acid and be labeled as high acetic-acid concentration with less overall flow (less load) to acetic acid unit 134, as compared to Options 1 and 2. Such could be implemented with a smaller acetic acid unit 134 having less heating (steam) and cooling demand as compared to Options 1 and 2. For Option 3 with the raw acetic acid 132 as high acetic-acid concentration, the concentration of acetic acid in the raw acetic acid 132 may be, for example, at least 1 wt %, at least 10 wt %, at least 20 wt %, at least 30 wt %, or in ranges of 1 wt % to 50 wt %, 1 wt % to 40 wt %, or 20 wt % to 50 wt %.

The flash drum 130 discharges gas 306 (including water vapor) overhead to the second flash drum 302. The gas 306 may be analogous to the gas 142 of preceding figures. The gas 306 is the effluent 104 minus the raw acetic acid 132. The gas 306 may include water vapor, residual acetic-acid vapor, and gases such as carbon dioxide, carbon monoxide, unreacted ethane, and other gases. The other gases may include, for example, relatively small amounts of methane or propane that entered the system 100 with the ethane gas 112. The gas 306 flows through the second cooler heat exchanger 304 that cools the gas 306, for example, to a temperature in a range of 30° C. to 80° C. The second cooler heat exchanger 304 condenses most of the acetic acid and water in the gas 306 remaining from the effluent 104.

Gas 142A discharges overhead from the second flash drum 302 and may be analogous to the gas 142 of preceding figures. The gas 142A may include water vapor, residual acetic-acid vapor, and gases such as carbon dioxide, carbon monoxide, propane, methane, unreacted ethane, etc.

The second flash drum 302 discharges a bottoms stream 308 that may be primarily water and that may be utilized as recycle water in implementations. For instance, the bottoms stream 308 may be combine with the bottoms stream of the acetic acid scrubber 144 to give the recycle water 150 sent to the ethane saturator tower 110. Thus, the bottoms stream from the acetic acid scrubber 144 incorporates the bottoms stream 308 (at higher temperature) from the second flash drum 302 to give the recycle water 150 to the ethane saturator tower 110. In implementations, due to higher temperature of recycle water 150 to the ethane saturator tower 110, the steam consumption at the circulation-water heater 118 may be less as compared to Options 1 and 2. Consequently, based on an energy balance, less cooling water may be utilized to cool down the reactor effluent 104.

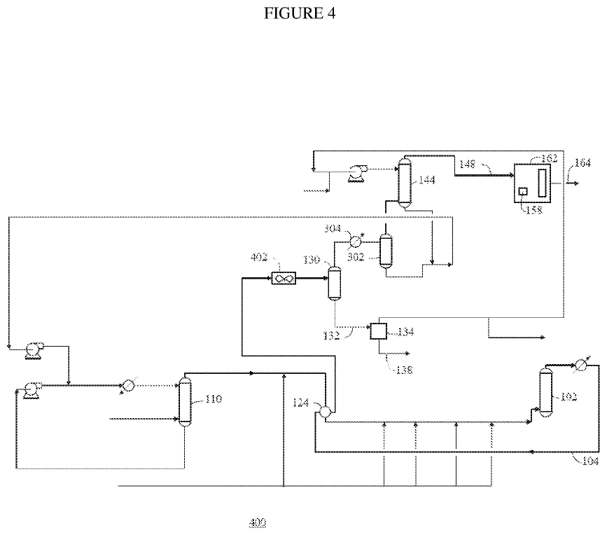

FIG. 4 is an ethylene production system 400 that is the same or similar as the ethylene production system 300 of FIG. 3 but with replacement of the first cooler heat exchanger 128 with air cooler 402. The overall cooling water demand would be less as compared to Option 3 and thus could beneficially lead to implementation with a smaller cooling water tower (for the ethylene production system) as compared to Option 3. FIG. 4 may be characterized as Option 4. For a description of text and reference numerals depicted in FIG. 4, see also the discussion of the preceding figures. Like reference numeral and designations in the various drawings indicate like elements.

The air cooler 402 is a heat exchanger that may be similar to the air cooler 202 of FIG. 2. The air cooler 402 cools (removes heat from) the effluent 104 with ambient air as the cooling medium (heat transfer fluid). The air cooler 202 may be operationally disposed between the feed heat exchanger 124 and the flash drum 130. The air cooler 402 may be fan heat exchanger that includes one or more fans. The air cooler 402 as a fan heat exchanger may include fins or a finned-tube bundle, and the like. The air cooler 402 may cool the effluent 104, for example, to a temperature in a range of 80° C. to 120° C. Therefore, the raw acetic acid 132 that discharges from the flash drum 130 may have a higher concentration, as with Option 3. The concentration of acetic acid in the raw acetic acid 134 may be, for example, at least 1 wt %, at least 10 wt %, at least 20 wt %, at least 30 wt %, or in ranges of 1 wt % to 50 wt %, 1 wt % to 40 wt %, or 20 wt % to 50 wt %, and the like. Such may be labeled as a high acetic-acid concentration in the raw acetic acid.

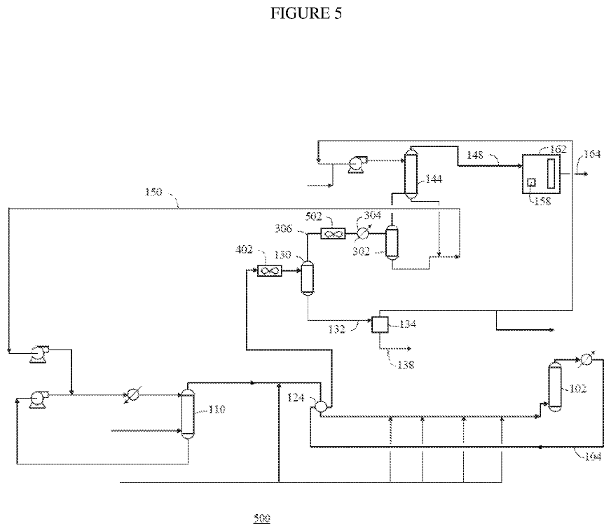

FIG. 5 is an ethylene production system 500 that is the same or similar as the ethylene production system 400 of FIG. 4 but with the addition of the second air cooler 502. FIG. 5 may be characterized as Option 5. For a description of text, designations, and reference numerals depicted in FIG. 5, see also the discussion of the preceding figures.

The second air cooler 502 may be the same or similar type of heat exchanger as the air cooler 402. The second air cooler 502 is operationally disposed between the flash drum 130 and cooler heat exchanger 304. The second cooler 502 cools (removes heat from) the gas 306 that discharges overhead from the first flash drum 130. The second air cooler 502 may cool the gas 306, for example, to a temperature in a range of 80° C. to 120° C. As with Options 3 and 4, the cooler heat exchanger 304 may cool the gas 306, for example, to a temperature in a range of 30° C. to 80° C. However, the heat removal from the gas 306 in Option 5 is shared between the second air cooler 502 and the cooler heat exchanger 304. Therefore, the cooling water demand by the cooler heat exchanger 304 may be generally less than in Options 3 and 4. Thus, the overall cooling water demand for the system 500 may be less than for the systems 300, 400. Such may beneficially lead to implementation with a smaller cooling water tower (to service the ethylene production system) for Option 5 as compared to Options 3 and 4. However, inclusion of the second air cooler 502 as an additional heat exchanger may result in further pressure drop between ODH reactor 102 and the PGC 158 potentially causing higher power demand by the PGC 158. Lastly, the raw acetic acid 132 may be at high acetic-acid concentration (as with Options 3 and 4), which is the raw acetic acid 132 having an acetic-acid concentration, for example, of at least 1 wt %.

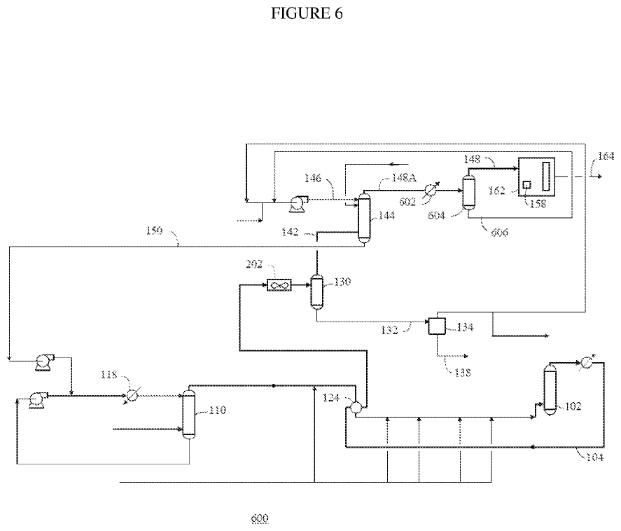

FIG. 6 is an ethylene production system 600 that is the same or similar as the ethylene production system 200 of FIG. 2 but with the removal of the cooler heat exchanger 128. A cooling heat exchanger 602 and flash tank 604 may also be added for the process gas from the overhead of the acetic acid scrubber 144, as discussed below. FIG. 6 may be characterized as Option 6. For a description of text, designations, and reference numerals depicted in FIG. 6, see also the discussion of the preceding figures.

Implementations for Option 6 may involve adjusting the outlet temperature of the effluent 104 from the air cooler 202 to achieve less than a specified threshold of amount or concentration of acetic acid in the process gas 148A at the overhead of the acetic acid scrubber 144. The amount or concentration of acetic acid in the process gas 148A may be correlative with (and directly proportional to) the temperature of the effluent 104 discharging from the air cooler 202. An increase in the temperature of the effluent 104 as discharged from the air cooler 202 may generally increase the amount or concentration of acetic acid in the process gas 148A. A decrease in the temperature of the effluent 104 as discharged from the air cooler 202 may generally decrease the amount or concentration of acetic acid in the process gas 148A.

The aforementioned specified threshold may be, for example, 50 ppmv of acetic acid. Again, in implementations via operation of the air cooler 202, the concentration in the process gas 148 at the overhead of the acetic acid scrubber 144 may be maintained less than the threshold. This may result in a slightly greater concentration of acetic acid in the raw acetic acid 132 as compared to Option 2. Less water relative to acetic acid in the raw acetic acid 132 may result in less heat demand at the acetic acid unit 134 compared to Option 2. A higher temperature of the effluent 104 may result in a higher temperature of gas 142, which may lead to a higher temperature at the bottom of acetic acid scrubber 144 and ultimately a higher temperature of the recycle water 150 to the ethane saturator tower 110. This could generally lead to less steam (e.g., low pressure steam) consumption at the circulation water heater 118 for ethane feed saturation as compared to Option 2.

Lastly, because the overhead temperature of the acetic acid scrubber 144 may be higher than in Option 2, the process gas may be cooled before reaching the PGC 158. In particular, a cooling heat exchanger 602 may be included to cool the process gas 148A discharged overhead from the acetic acid scrubber 144. The cooling heat exchanger 602 may utilize water (e.g., cooling tower water) as the heat transfer fluid (cooling medium). The cooling heat exchanger 602 may condense nearly all the acetic acid (and water vapor) carried over from the acetic acid scrubber 144 in the process gas 148A before going through PGC 158. A flash tank 604 (vessel) may be included to recover the condensed fluid 606 including the acetic acid and water. The condensed fluid may be utilized for scrubbing water 146 as depicted. The process gas 148 may discharge overhead from the flash tank 604 for processing in the downstream equipment 162.

FIG. 6 (ethylene production system 600) may also be characterized as Option 7 but with operating differences compared to Option 6. In Option 7, operation of the air cooler 202 may be adjusted to give a discharged effluent 104 temperature that gives a high acetic-acid concentration (e.g., at least 1 wt %) in the raw acetic acid 132, as with Options 3-5. This may reduce heating and cooling demand at acetic acid unit 134. At these higher temperatures in the flash drum 130, the concentration of acetic acid in the process gas 148A at the overhead of the acetic acid scrubber 144 may be at least the aforementioned threshold, e.g., at least 50 ppmv or in a range of 50 ppm to 200 ppmv. The overhead of the acetic acid scrubber 144 may warmer in Option 7 than in Option 6. The cooling heat exchanger 602 may condense (knock out) nearly all acetic acid in the process gas stream 148 before the process gas stream 148 enters the PGC 158. In addition, the recycle water temperature 150 to the ethane saturator tower 110 may be higher which could result in less steam [e.g., low pressure (LP) steam] demand for ethane (or feed) saturation, such as at the circulation water heater 118.

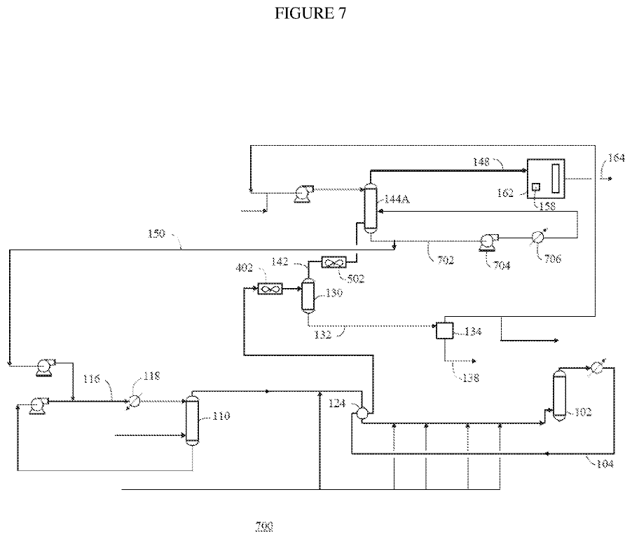

FIG. 7 is an ethylene production system 700 that is the same or similar as the ethylene production system 500 of FIG. 5 but with elimination of the cooler heat exchanger 304 and the second flash drum 302. Elimination of cooler heat exchanger 304 and second flash drum 302 may result in higher suction pressure at the PGC 158 and therefore lower power demand of the PGC 158 as compared to Option 5. FIG. 7 (in contrast to FIG. 5) also includes reconfiguration of the acetic acid scrubber 144 incorporating a quenching section (also called quench section). The acetic acid scrubber as so reconfigured may thus become a quench/acetic acid scrubber 144A, which is an acetic acid scrubber having a quenching section.

FIG. 7 (ethylene production system 700) may be characterized as Option 8. Option 8 may be directed, in part, to reducing pressure drop of processing the effluent 104. Option 8 may be an effort to reduce such pressure drop in particular with respect to Option 5. For a description of text, designations, and reference numerals depicted in FIG. 7, see also the discussion of the preceding figures.

The overhead gas 142 discharged from the flash drum 130 flows through the second air cooler 502 to the quench/acetic acid scrubber 144A. In the illustrated embodiment of FIG. 7, there is no cooler heat exchanger or second flash drum operationally disposed between the second air cooler 502 and the quench/acetic acid scrubber 144A.

A portion of the bottoms streams from the quench/acetic acid scrubber 144A may be sent as recycle water 150 for the liquid water 116 feed to the ethane saturator tower 110. The remaining portion of the bottoms stream may be utilized as quench water 702 for the quenching section (e.g., lower portion) of the quench/acetic acid scrubber 144A. The quench water 702 may be returned via a conduit and introduced into the quench/acetic acid scrubber 144A at or just above the quenching section. Motive force for flow (recirculation) of the quench water 702 may be provided by circulation pump 704 (e.g., a centrifugal pump). A quench water cooler 706 heat exchanger utilizing water (e.g., cooling tower water) as a heat transfer medium may cool the quench water 702. The quench water cooler 706 may be, for example, a shell-and-tube heat exchanger, a plate-and-frame heat exchanger, a plate-fin heat exchanger, etc.

To reconfigure the acetic acid scrubber 144 (see FIGS. 1-6) to become the quench/acetic acid scrubber 144A depicted in FIG. 7, a lower section of the acetic acid scrubber becomes a quench section with quench water 702 circulating from a bottom of the of acetic acid scrubber (tower) to the lower section (portion) of the acetic acid scrubber. The lower section as a quenching section may include random packing, structured packing, or trays, or any combinations thereof. In implementations, a chimney tray may be disposed between the quenching and scrubbing sections. Again, the quenching and scrubbing sections may be trayed or packed. In some implementations, the internals in the quenching section may be similar to internals of the remainder of the scrubber.

The lower section (quench section) may include, for example, spray nozzles or a distributor at an upper portion of the lower section for receiving and discharging the quench water 702. The quench water 702 circulation rate and temperature for the quench section may be adjusted to achieve same or similar acetic-acid concentration and temperature at the overhead of scrubber as in Option 1. An alternative design is for the quench and acetic acid scrubber to be separate towers. In other words, the alternate configuration is to retain the acetic acid scrubber 144 (as in FIGS. 1-6) but add a quench tower vessel operationally upstream of the acetic acid scrubber to process the process gas in route to the PGC 158. A purpose of the quenching section (or separate quench tower in the alternate configuration) may be to cool the process gas 142 and remove more acetic acid and water from the process gas 142.

For Option 8, implementation of the quench/acetic acid scrubber 144A as depicted in FIG. 7 (and also the alternate configuration) may result in higher temperature of the recycle water 150 (from the quench/acetic acid scrubber 144A to the ethane saturator tower 110) due to the heat removal by the quenching section of the quench/acetic acid scrubber 144A. This is so because of a higher temperature of the recycle water 150 may result in less steam consumption at the circulation water heater 118 as compared to Option 5.

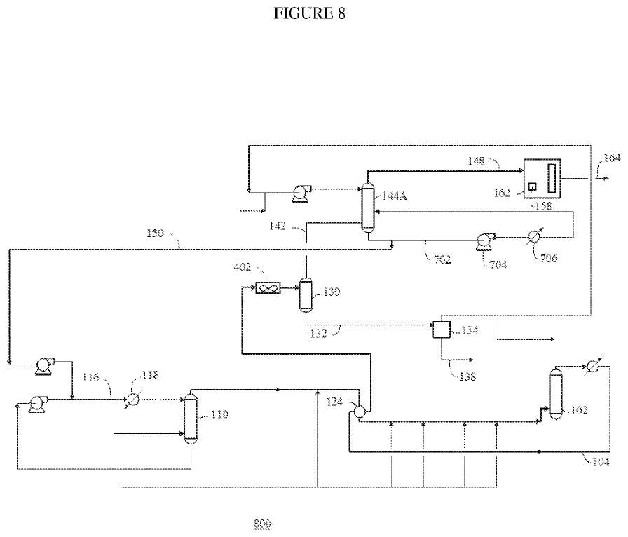

FIG. 8 is an ethylene production system 800 that is the same or similar as the ethylene production system 700 of FIG. 7 (Option 8) but with elimination of the second air cooler 502. FIG. 8 may be characterized as Option 9. For a description of text, designations, and reference numerals depicted in FIG. 8, see also the discussion of the preceding figures.

In FIG. 8 (Option 9), elimination of the second air cooler 502 may result in higher suction pressure at the PGC 158 and thus lower power consumption by the PGC 158 as compared to Option 8. In Option 9, the quench water 702 circulation rate associated with the quench section of the quench/acetic acid scrubber 144A may be increased to advance removal of the extra heat load shifted from the eliminated air cooler 502. As compared to Option 8, this may add more load of cooling medium (e.g., cooling tower water) through quench water cooler 706, but would increase temperature of recycle water 150 from the bottom of quench/acetic acid scrubber 144A and therefore reduce steam consumption by the circulation water heater 118 (for ethane saturation).

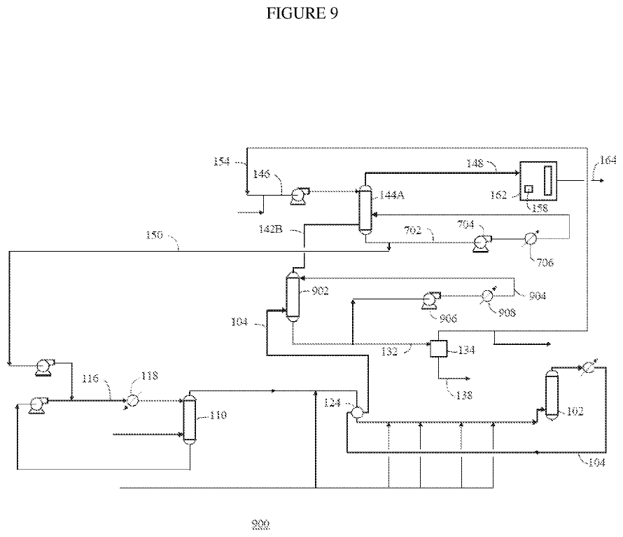

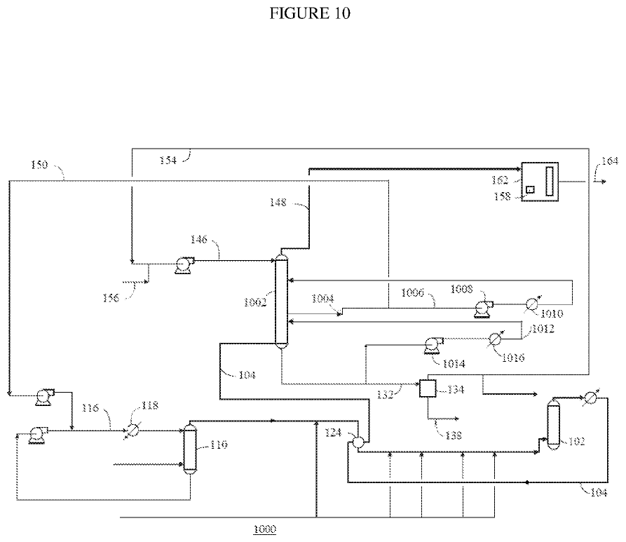

FIG. 9 and FIG. 10 are Option 10 that further reduces pressure drop (in comparison to Option 9) between the ODH reactor 102 and the PGC 158 by eliminating the air cooler 402 and the flash drum 130. This may generally result in the highest suction pressure and lowest power consumption by PGC among the example configurations described and tabulated below as example Options 1-22. Cooling water demand may increase to remove the extra shifted heat-load from the eliminated air cooler 402. This may add more load to cooling tower through quench water coolers compared to Option 9.

FIG. 9 is an ethylene production system 900 that includes a quench tower 902 (vessel) to receive the effluent 104 from the feed heat exchanger 124. The quench tower 902 may be a vessel (column) with a vertical orientation and that may include spray nozzles, random packing, structured packing, or trays, or any combinations thereof. The quench tower 902 may cool (remove heat from) the effluent 104. The quench tower 902 may remove water and acetic acid from the effluent 104.

The quench tower 902 may discharge overhead the gas 142B as feed to the quench/acetic acid scrubber 144A. The gas 142B may generally be the effluent 104 minus the water and acetic acid removed by the quench tower 902.

The quench tower 902 may discharge a bottoms stream having the removed water and removed acetic acid. A portion of the bottoms stream may be sent as raw acetic acid 132 to the acetic acid unit 134. The remainder of the bottoms stream may be circulated as quench water 904 for the quench tower 902. A circulation pump 906 (e.g., centrifugal pump) may provide motive force for flow (circulation) of the quench water 904.

A quench water cooler 908 may cool (remove heat from) the quench water 904 in the circulation. The quench water cooler 908 may utilize water (e.g., cooling tower water) as the heat transfer fluid (cooling medium). The quench water cooler 908 may be, for example, a shell-and-tube heat exchanger, a plate-and-frame heat exchanger, or a plate-fin heat exchanger, and so on. The amount of heat (duty) removed from the effluent 104 by the quench tower 902 in conjunction with the quench water cooler 908 may be correlative with the heat removal from the effluent 104 by heat exchangers depicted in previous figures downstream of the feed heat exchanger 124.

FIG. 10 is an ethylene production system 1000 that includes a quench/acetic acid scrubber 1002 to receive the effluent 104 from the feed heat exchanger 124. The quench/acetic acid scrubber 1002 may have two quenching sections: a quenching mid-section and a quenching lower section. The quench/acetic acid scrubber 1002 may be a vertical vessel (column, tower) including random packing, structured packing, or trays, or any combinations thereof. In some implementations, spray nozzles or similar devices may be included at an upper part of (or above) each quenching section. A purpose of the quenching sections is for the quench/acetic acid scrubber 1002 to remove heat from the effluent 104.

In operation, water 1004 is withdrawn at or below a bottom part of the quenching mid-section. A portion of the water 1004 is sent as recycle water 150. Another portion of the water 1004 is recirculated as quench water 1006 to the quench/acetic acid scrubber 1002 at or above an upper part of the quenching mid-section. A circulation pump 1008 (e.g., centrifugal pump) may pump (provide motive force for flow of) the quench water 1006. A quench water cooler 1010 may remove heat from the quench water 1006. The quench water cooler 1010 may be, for example, a shell-and-tube heat exchanger, a plate-and-frame heat exchanger, or a plate-fin heat exchanger, and the like. The quench water cooler 1010 may utilize water (e.g., cooling tower water) as the cooling medium.

The quench/acetic acid scrubber 1002 discharges a bottom stream having acetic acid and water removed from the effluent 104 via the scrubbing and quenching in the quench/acetic acid scrubber 1002. A portion of the bottoms stream may be sent as raw acetic acid 132 to the acetic acid unit 134. Another portion of the bottoms stream may be recirculated as quench water 1012 back to the quench/acetic acid scrubber 1002 at or above an upper part of the quenching lower-section. This may be similar to the operation associated with the quench tower 902 FIG. 9. The circulation pump 1014 (e.g., centrifugal pump) may provide motive force for flow (circulation) of the quench water 1012. The quench water cooler 1016 may cool (remove heat from) the quench water 1012 in the circulation. The quench water cooler 1016 may utilize water (e.g., cooling tower water) as the heat transfer fluid (cooling medium). The quench water cooler 1016 may be, for example, a shell-and-tube heat exchanger, a plate-and-frame heat exchanger, or a plate-fin heat exchanger, and so on.

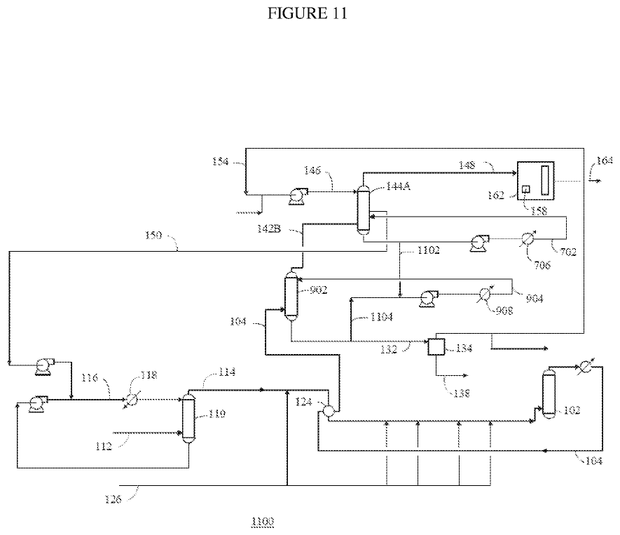

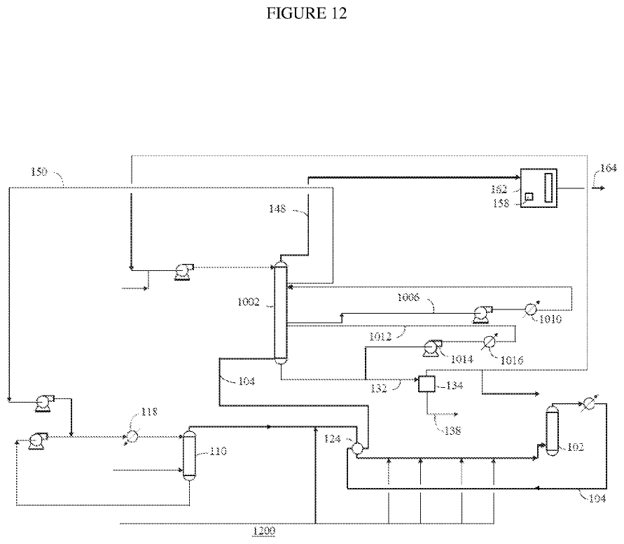

The lower quenching section of the quench/acetic acid scrubber 1002 (and quenching in the quench tower 902 in the FIG. 9) is where the concentrated raw acetic acid is condensed, collected, and sent to acetic acid unit 134. The mid-section quench in the quench/acetic acid scrubber 1002 [and lower section (quenching section) of the quench/acetic acid scrubber 144A in FIG. 9] is where the final quenching is carried out and water is collected/sent as recycle water 150. The scrubbing section, which is the top section in the quench/acetic acid scrubber 1002 (or top section of the quench/acetic acid scrubber 144A in FIG. 9) receives scrubbing water for final removal of remainder of acetic acid. The water from this scrubbing section goes down the tower and mixes with water circulation of the mid-section in the quench/acetic acid scrubber 1002 [or of the lower section (quenching section) in the quench/acetic acid/scrubber 144A in FIG. 9]. This last point is a difference between FIGS. 9, 10 versus FIGS. 11, 12.

As mentioned, the quenching sections and remainder of the quench/acetic acid scrubber 1002 may remove water and acetic acid from the effluent 104. The source of the scrubbing liquid 146 that enters an upper portion of the quench/acetic acid scrubber 1002 may be the same or similar as with the acetic acid scrubber 144 and the quench/acetic acid scrubber 144A depicted in previous figures. The scrubbing liquid 146 may be the combination of water 154 from the acetic acid unit 134 and condensate water 156 from the PGC 158. The quench/acetic acid scrubber 1002 may discharge overhead the process gas 148 as feed to PGC 158.

As indicated, the quench/acetic acid scrubber 1002 may cool (remove heat from) the effluent 104. The amount of heat (duty) removed from the effluent 104 by the quench/acetic acid scrubber 1002 in conjunction with the quench water cooler 1016 and quench water cooler 1010 may be correlative with the heat removal from the effluent 104 by heat exchangers depicted in previous figures downstream of the feed heat exchanger 124.

The circulation rate of the quench water 1012 and process temperature of the quench water cooler 1016 for the quenching lower section may be set (specified) to achieve high concentration (e.g., at least 1 wt %) of acetic acid in the raw acetic acid 132 to the acetic acid unit 134. The quenching mid-section circulation rate and temperature of the quench water 1006 may be adjusted to achieve an acetic-acid concentration and temperature at the overhead process gas 148 similar to that of the acetic acid scrubber 144 of FIG. 1 for Option 1. Again, a portion of the water 1004 outlet from the quenching mid-section may be circulated as quench water 1006 and the rest is recycled back as recycle water 150 to ethane saturator tower 110.

FIG. 11 is an ethylene production system 1100 that is the same or similar as the ethylene production system 900 of FIG. 9 but with differences with respect to: (1) the source of the quench water 904 for the quench tower 902; and (2) withdrawal of water for recycle water 150 to the ethane saturator tower 110. FIG. 11 may be characterized as Option 11. For a description of text, designations, and reference numerals depicted in FIG. 11, see also the discussion of the preceding figures.

The water for the recycle water 150 to the ethane saturator tower 110 is water from the scrubbing section of the quench/acetic acid scrubber 144A. The recycle water 150 is taken from the scrubbing section (top section) of the quench/acetic acid scrubber 144A.

The quench water 904 for the quench tower 902 is a combination of water 1104 (raw acetic acid) from the bottom of the quench tower 902 and water 1102 from the bottom of the quench section of the quench/acetic acid scrubber 144A. The raw acetic acid from the bottom of the quench tower 902 has lower acetic acid concentration compared to the raw acetic acid in FIGS. 9 and 10. The raw acetic acid in FIG. 11 may be similar to that in Option 1 with respect to concentration of acetic acid. Thus, the quench water 904 for the quench tower 902 is a combination of a portion 1102 of the bottoms stream from the quench/acetic acid scrubber 144A and a portion 1104 of the bottoms stream from the quench tower 902.

This reconfiguration with respect to the recycle water 150 and the quench water 904 can lead to lower concentration (e.g., less than 1 wt %) of acetic acid in the raw acetic acid 132 as compared to Option 10.

Referring to FIG. 9, with controlled temperature and water circulation on the quench tower 902 in FIG. 9, concentration of acetic acid in the raw acetic acid 132 is generally low because the bulk (majority) of water in the effluent 104 is condensed along with the bulk (majority) of the acetic acid in the effluent 104 being condensed. The overhead gas 142B from the quench tower includes the remaining portions of acetic acid and water from the effluent 104. In the quench/acetic acid scrubber 144A, quenching and scrubbing remove generally this remainder of acetic acid and water. Therefore, the recycle water 150 to ethane saturator tower 110 will typically include less acetic acid.

Returning to FIG. 11, the recycle water 150 is provided from a top section (scrubbing section) of the quench/acetic acid scrubber 144A. The gas 142B that enters this section generally does not contain significant acetic-acid concentration and, therefore, there is typically not a significant amount of acetic acid in the recycle water 150 to the ethane saturator tower 110. The gas 142B as discharged from the overhead of the quench tower 902 includes acetic acid and enters the lower section of the quench/acetic acid scrubber 144A where the bulk of the acetic acid and water in the gas 142B is condensed and sent to the quench tower 902 (though this stream 1102 generally has low concentration of acetic acid). In all, similar to Option 1, most (e.g., nearly all) of the acetic acid and water in the effluent 104 is withdrawn from the effluent 104 as raw acetic acid. Again, raw acetic acid 132 may be processed in the acetic acid unit 134.

For a comparison of FIG. 11 versus FIG. 9, the recycle water 150 to the ethane saturator tower 110 in FIG. 9 typically has a greater concentration of acetic acid than the recycle water 150 to the ethane saturator tower 110 in FIG. 11. The raw acetic acid 132 in FIG. 9 typically has a greater concentration of acetic acid than the raw acetic acid 132 in FIG. 11.

For FIG. 11 (Option 11), the lower concentration of acetic acid in the raw acetic acid 132 could lead to increased heating and cooling consumption in the acetic acid unit 134, which would generally be greater for this Option 11 as compared to Options 9 and 10. In addition, because the recycle water 150 has a lower temperature, the steam requirement at the circulation water heater 118 for the ethane saturator tower 110 is higher than Option 10. An advantage of Option 11 may be a lower concentration of acetic acid in the recycle water 150. Option 11 is better than Option 1 in terms of less energy consumption. Option 11 consumes less energy than Option 1.

FIG. 12 is an ethylene production system 1200 that is same or similar as the ethylene production system 1000 of FIG. 10, except for the source of the recycle water 150. For a description of text, designations, and reference numerals depicted in FIG. 12, see also the discussion of the preceding figures. FIG. 12 (ethylene production system 1200) is a variation for Option 11 that does not employ separate quench tower 902.

The recycle water 150 is withdrawn from the top section (scrubbing section) of the quench/acetic acid scrubber 1002. The recycle water 150 is taken from the bottom of scrubbing section of quench/acetic acid scrubber 1002. A chimney tray may be disposed between the mid-section and top section (scrubbing). Again, water from the scrubbing section can be recycled back to ethane saturator tower 110. A chimney tray between the lower section and middle section of the quench/acetic acid scrubber 1002 may be optional.

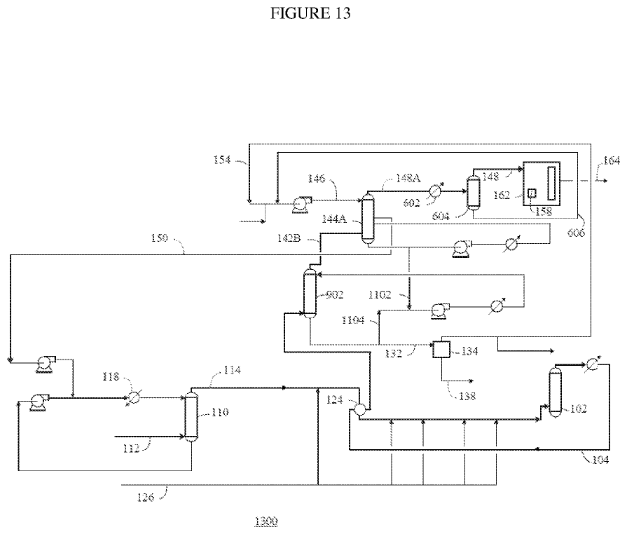

FIG. 13 is an ethylene production system 1300 that is the same or similar as the ethylene production system 1100 of FIG. 11 but with the addition of cooling heat exchanger 602 and flash tank 604 for the process gas upstream of the PGC 158, as was similarly done in FIG. 6. FIG. 13 may be characterized as Option 12. For a description of text, designations, and reference numerals depicted in FIG. 13, see also the discussion of the preceding figures.

Option 12 is different than Option 11 in that in Option 12, quench water 702 circulation rate for the quench/acetic acid scrubber 144A is adjusted (lowered) to achieve less than a specified threshold value (e.g., 50 ppmv) of acetic acid at the overhead of the quench/acetic acid scrubber 144A (e.g., in the process gas 148A). This may result in higher temperature of process gas 148A at the overhead. Thus, because the overhead temperature of the quench/acetic acid scrubber 144A may be higher than in FIG. 11 (Option 11), the process gas 148A may be cooled before reaching the PGC 158. In particular, a cooling heat exchanger 602 may be included to cool the process gas 148A discharged overhead from the quench/acetic acid scrubber 144A. The cooling heat exchanger 602 may utilize water (e.g., cooling tower water) as the heat transfer fluid (cooling medium). The cooling heat exchanger 602 may condense nearly all the acetic acid (and water vapor) carried over from the acetic acid scrubber 144 in the process gas 148A before going through PGC 158. A flash tank 604 (vessel) may be included to recover the condensed fluid 606 including the acetic acid and water. The condensed fluid 606 may be utilized for scrubbing water 146 as depicted. The process gas 148 may discharge overhead from the flash tank 604 for processing in the downstream equipment 162. Lastly, an adjusted (e.g., lower) quench water 702 circulation could lead to higher temperature of recycle water to the ethane saturator tower 110 and thus less steam (e.g., LP steam) demand at the circulation water heater 118 for ethane saturation as compared to Option 11.

FIG. 14 is an ethylene production system 1400 that is the same or similar as the ethylene production system 800 of FIG. 8 (Option 9) but with the addition of an extract cross-exchanger 1402 (heat exchanger) associated with the acetic acid unit 134. FIG. 14 may be labeled as Option 13. For a description of text, designations, and reference numerals depicted in FIG. 14, see also the discussion of the preceding figures.

The acetic acid unit 134 includes an extractor column 1404 to utilize solvent to remove acetic acid from the raw acetic acid 132, a water stripper column 1406 to process raffinate from the extractor column 1404 to recover water, and a solvent recovery column 1408 to remove the solvent from the acetic acid discharged from the extractor column 1404 to give the acetic acid product 138. The acetic acid unit 134 receives the raw acetic acid 132, as discussed. The raw acetic acid 132 can be primarily water.

In the illustrated embodiment, the raw acetic acid 132 is fed to the extractor column 1404. The raw acetic acid 132 may be introduced at an upper portion of the extractor column 1404 and flow downward through the extractor column 1404.

The extractor column 1404 is a vessel generally having a vertical orientation. The extractor column 1404 may be a liquid-liquid extraction column. The extractor column 1404 may have packing (random or structured) or trays (e.g., sieve trays), and moving components such as impellers to better contact liquid-liquid phases. If packing is employed, the packing may be metal (e.g., stainless steel) or plastic.

In operation, the extractor column 1404 utilizes a solvent 1410 to extract acetic acid from the raw acetic acid 132. The solvent 1410 may generally be immiscible with water and thus typically does not remove a significant amount of water from the raw acetic acid 132. The solvent 1410 may be, for example, n-butanol, isobutanol, amyl alcohol, or ethyl acetate, methyl tert-butyl ether (MTBE), and so forth. The solvent 1410 may be introduced at a bottom portion of the extractor column 1404 and flow upward through the extractor column 1404 in a countercurrent flow with the raw acetic acid 132 flowing downward through the extractor column 1404. The solvent 1410 removes (absorbs, extracts) acetic acid from the raw acetic acid 132. The packing or trays (and moving parts) in the extractor column 1404 facilitates mass transfer of the acetic acid into the solvent 1410.

Extract 1412 including the solvent 1410 and the removed (absorbed, extracted) acetic acid (having a small amount of water) discharges overhead from the extractor column 1404 through an extract heater 1414 (heat exchanger). The extract heater 1414 heats the extract 1412. The heating medium may be, for example, steam. The extract heater 1414 may be a shell-and-tube heat exchanger, a plate heat exchanger, or a plate-fin heat exchanger, or other type of heat exchanger.

The extract 1412 is then routed through an extract cross-exchanger 1402 to heat the extract 1412 with the quench water 702 as a heating medium. The extract 1412 may be routed through an extract cross-exchanger 1402 to cool the quench water 702 (remove heat from quench water 702 into the extract 1412), with the extract 1412 as a cooling medium. This heating of the extract 1412 (in addition to the heat added by the extract heater 1414) may reduce the steam demand for the reboiler heat exchanger of the solvent recovery column 1408 as compared to Option 9 (FIG. 8). The extract 1412 can be partially vaporized or fully vaporized (and the vapor may be superheated) in the extract cross-exchanger 1402. In implementations, the extract cross-exchanger 1402 may be physically located in the acetic acid unit 134 and/or may be characterized as a component of the acetic acid unit 134.

The extract cross-exchanger 1402 may be, for example, a shell-and-tube heat exchanger, plate heat exchanger, or a plate-fin heat exchanger, and the like. The extract 1412 and the quench water 702 may be routed through either side of the extract cross-exchanger 1402, respectively. For instance, the cross-exchanger as a shell-and-tube heat exchanger may be configured such that the extract 1412 flows through the tubes (tube bundle) and the quench water 702 flows through the shell. Alternatively, the exchanger may be configured such that the quench water 702 flows through the tubes and the extract 1412 flows through the shell.

The extractor column 1404 discharges raffinate 1416 as a bottoms stream from a bottom portion of the extractor column 1404. The raffinate 1416 includes the majority or bulk (e.g., nearly all) of the water from the raw acetic acid 132. The raffinate 1416 is primarily water. The raffinate 1416 may include trace amounts of organic compounds (e.g., solvent 1410, acetic acid, etc.).

The raffinate 1416 is discharged from the extractor column 1404 to the water stripper column 1406 to recover (increase purity of) the water. The water stripper column 1406 (vessel) is a distillation column including distillation trays or packing and may be associated with an overhead condenser heat exchanger (and decanter to separate water phase from solvent phase) and a reboiler heat exchanger (or direct steam injection to the bottom as heat source). The distillation column system may include a receiver vessel or reflux drum to receive condensed liquid from the overhead condenser. In operation, the water stripper column 1406 may separate the trace amounts of organic compounds from the raffinate 1416 and discharge a bottoms streams having water with the trace amount of organic compounds as the liquid water 1418. The water stripper column 1406 may discharge water vapor and the majority of organic compounds overhead that is condensed into a decanter for solvent and water separation. A portion of the water 1418 may be forwarded as water product 140. Another portion 154 of the water 1418 may be utilized as scrubbing water 146 for the quench/acetic acid scrubber 144A.

The solvent recovery column 1408 receives the extract 1412 from the extract cross-exchanger 1402. The solvent recovery column 1408 may be a distillation column that separates solvent 1410 from the extract 1412 to give the acetic acid product 138. The separated solvent 1410 may be sent to the extractor column 1404. The distillation column is a vessel having distillation trays or packing and operates with a reboiler heat exchanger and an overhead condenser heat exchanger (with overhead decanter to separate the condensed overhead liquid into water phase and solvent phase).

The extract 1412 may be introduced as a side feed (e.g., upper portion) of the solvent recovery column 1408. The acetic acid product 138 may be a bottoms stream discharged from the solvent recovery column 1408. The solvent 1410 may be discharged overhead from the solvent recovery column 1408 and then condensed.

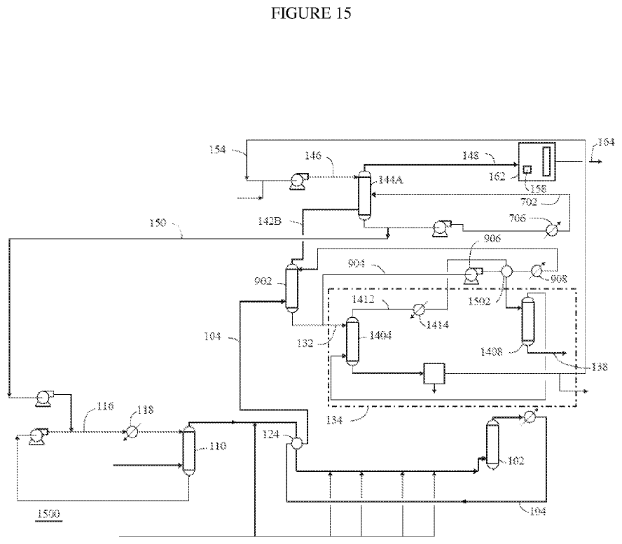

FIG. 15 is an ethylene production system 1500 that is the same or similar as the ethylene production system 900 of FIG. 9 but with the addition of an extract cross-exchanger 1502 (heat exchanger) associated with the acetic acid unit 134. FIG. 15 may be labeled as Option 14. For a description of text, designations, and reference numerals depicted in FIG. 15, see also the discussion of the preceding figures.

The extract cross-exchanger 1502 may be similar to the extract cross-exchanger 1402 of FIG. 14. The extract cross-exchanger 1502 may be, for example, a shell-and-tube heat exchanger or a plate-fin heat exchanger, and the like.

The extract cross-exchanger 1502 is a similar implementation as with the extract cross-exchanger 1402 in FIG. 14, except that the extract cross-exchanger 1502 is implemented on a different quench-water circulation loop. The extract cross-exchanger 1502 utilizes quench water 904 as the heating medium to heat the extract 1412. In the illustrated embodiment of FIG. 15, the extract cross-exchanger 1502 is operationally disposed between the circulation pump 906 and the quench water cooler 908.

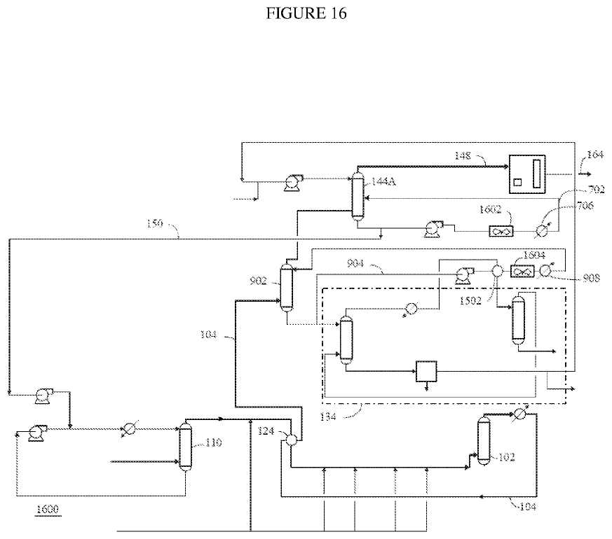

The ethylene production system 1500 includes the extract cross-exchanger 1502 to heat the extract 1412 with the quench water 904 as a heating medium, and to cool the quench water 904 (remove heat from quench water 904 into the extract 1412), with the extract 1412 as a cooling medium. This heating of the extract 1412 by the extract cross-exchanger 1502 (in addition to the heat added by the extract heater 1414) may reduce the steam demand for the reboiler heat exchanger of the solvent recovery column 1408 as compared to FIG. 9 (Option 10). The extract 1412 can be partially vaporized or completely vaporized (and the vapor can be superheated) in the extract cross-exchanger 1502. This is so with the previous FIG. 14 and in subsequent FIGS. 16, 17, and 20-23. In implementations, the extract cross-exchanger 1502 may be physically located in the acetic acid unit 134 and/or may be characterized as a component of the acetic acid unit 134.

FIG. 16 is an ethylene production system 1600 that is the same or similar as the ethylene production system 1500 of FIG. 15 but with the addition of two air coolers 1602 and 1604 to cool quench water 702 and quench water 904, respectively. FIG. 16 may be labeled as Option 15. For a description of text, designations, and reference numerals depicted in FIG. 16, see also the discussion of the preceding figures.

The air cooler 1602 is disposed along the quench water 702 circulation loop (upstream of the quench water cooler 706) to cool the quench water 702. Similarly, the air cooler 1604 is disposed along the quench water 904 circulation loop (upstream of the quench water cooler 908) to cool the quench water 904. The air cooler 1602 cools down the quench water 702 to 80° C. or less before the quench water 702 is cooled against cooling water in the quench water cooler 706. Likewise, the air cooler 1604 cools down the quench water 904 to 80° C. or less before the quench water 904 is cooled against cooling water in the quench water cooler 908. In embodiments, the addition of these two air coolers 1602 and 1604 may be implemented with the ethylene production system 1600 having a cooling water system (e.g., including a cooling water tower) that might be less capital intensive but with similar or slightly higher energy demand as compared to that in the ethylene production system 1500 of FIG. 15 (Option 14).

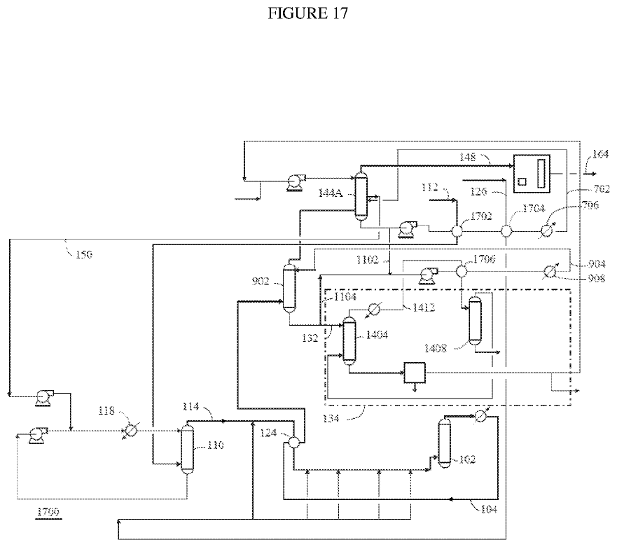

FIG. 17 is an ethylene production system 1700 that is the same or similar as the ethylene production system 1100 of FIG. 11 but with the addition of an ethane cross-exchanger 1702, an oxygen cross-exchanger 1704, and an extract cross-exchanger 1706. FIG. 17 may be labeled as Option 16. For a description of text, designations, and reference numerals depicted in FIG. 17, see also the discussion of the preceding figures.

The ethane cross-exchanger 1702 heats the ethane gas 112 that is fed to the ethane saturator tower 110. The oxygen cross-exchanger 1704 heats the oxygen gas 126 that is added to the saturated ethane 114. The ethane cross-exchanger 1702 and the oxygen cross-exchanger 1704 are each a heat exchanger that may be, for example, a plate-fin heat exchanger or a shell-and-tube heat exchanger, and the like. The quench water 702 is the heating medium for both the ethane cross-exchanger 1702 and the oxygen cross-exchanger 1704. In implementations, the ethane cross-exchanger 1702 and the oxygen cross-exchanger 1704 are each operationally disposed in the quench-water 702 circulation loop upstream of the quench water cooler 706, as depicted.

The ethane gas 112 and oxygen gas 126 feed preheating with the cross-exchangers 1702 and 1704, may reduce the steam demand at the circulation water heater 118 for ethane feed saturation in the ethane saturator tower 110. However, the amount of heat recovery may be relatively low or insignificant compared to overall steam demand of the circulation water heater 118 for the ethane feed saturation. Nevertheless, a value on heat demand reduction is realized.

The extract cross-exchanger 1706 heats the extract 1412 discharged from the extractor column 1404 of the acetic acid unit 134. The extract 1412 can be partially vaporized or completely vaporized (and the vapor may be superheated) in the extract cross-exchanger 1706. The quench water 904 is the heating medium. The extract cross-exchanger 1706 may be operationally disposed in the quench-water 904 circulation loop upstream of the quench water cooler 908, as depicted. In implementations, the extract cross-exchanger 1502 may be physically located in the acetic acid unit 134 and/or may be characterized as a component of the acetic acid unit 134.

The extract 1412 may flow from the extract cross-exchanger 1706 to the solvent recovery column 1408 of the acetic acid unit 134. The heating of the extract 1412 by the extract cross-exchanger 1706 may reduce the steam demand for the reboiler heat exchanger of the solvent recovery column 1408 as compared to FIG. 11 (Option 11). The extract cross-exchanger 1706 may be similar to the extract cross-exchangers previously discussed. The extract cross-exchanger 1502 may be, for example, a shell-and-tube heat exchanger, plate heat exchanger, or a plate-fin heat exchanger, or other type of heat exchanger.

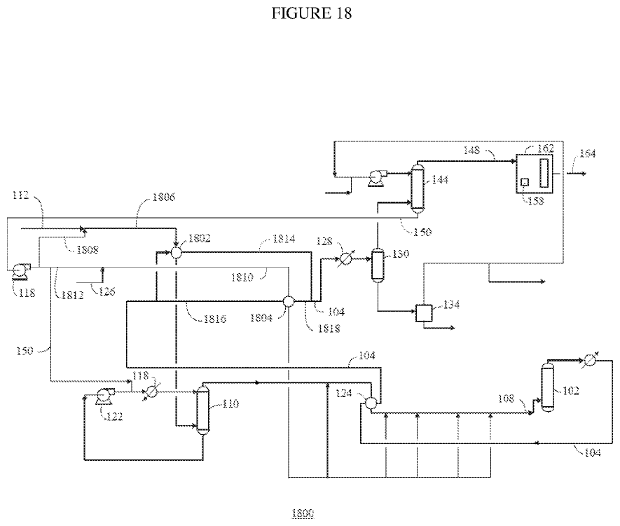

FIG. 18 is an ethylene production system 1800 that is the same or similar as the ethylene production system 100 of FIG. 1 but with an ethane cross-exchanger 1802 and an oxygen cross-exchanger 1804, and associated water addition for partial saturation. FIG. 18 may be labeled as Option 17. For a description of text, designations, and reference numerals depicted in FIG. 18, see also the discussion of FIG. 1.

The ethane cross-exchanger 1802 heats a mixture 1806 of the ethane gas 112 and the recycle water 1808. The mixture 1806 (as heated) downstream of the ethane cross-exchanger may be labeled as partially-saturated ethane that is fed to the ethane saturator tower 110. Thus, instead of feeding the ethane gas 112 directly as in FIG. 1, the ethane gas 112 is first partially saturated with the recycle water 1808 prior to introduction to the ethane saturator tower 110. In the illustrated embodiment, the recycle water 1808 is a portion of the recycle water 150 from the bottoms of the acetic acid scrubber 144.

The oxygen cross-exchanger 1804 heats a mixture 1810 of the oxygen gas 126 and the recycle water 1812. The mixture 1810 (as heated) downstream of the oxygen cross-exchanger may be labeled as partially-saturated oxygen that is added (injected) to the saturated ethane 114 at one or more addition points. Thus, instead of adding the oxygen gas 126 directly as in FIG. 1, the oxygen gas 126 is first partially saturated with the recycle water 1812 prior to introduction into the conduit conveying the saturated ethane 114. In the illustrated embodiment, the recycle water 1812 is a portion of the recycle water 150 from the bottoms of the acetic acid scrubber 144. (The remainder of the recycle water 150 may flow through the circulation water heater 118 to the ethane saturator tower 110.)

The ethane cross-exchanger 1802 and the oxygen cross-exchanger 1804 may each be a shell-and-tube heat exchanger, plate heat exchanger, or a plate-fin heat exchanger, or other type of heat exchanger. The ethane cross-exchanger 1802 and the oxygen cross-exchanger 1804 may utilize the effluent 104 as the heating medium, either in series or in parallel as depicted.

In the illustrated implementation, the ethane cross-exchanger 1802 and the oxygen cross-exchanger 1804 receive the effluent 104 downstream of the feed heat exchanger 124. A portion 1814 of the effluent 104 is fed to the ethane cross-exchanger 1802. The remaining portion 1816 of the effluent 104 is fed to the oxygen cross-exchanger 1804. The portions 1814 and 1816 may be divided, for example, via a pipe tee or other piping fitting. Thus, the conduit conveying the effluent 104 may discharge to two conduits conveying the portions 1814 and 1816, respectively. A control valve may be disposed on one of the two conduits. Other arrangements or configurations for dividing the effluent 104 into the portions 1814 and 1816 are applicable.

The portions 1814 and 1816 of the effluent 104 may be combined to give the effluent 104 going forward as cooled by the ethane cross-exchanger 1802 and the oxygen cross-exchanger 1804. The effluent 104 (as cooled) may flow through the cooler heat exchanger 128 (for additional cooling) to the flash drum 130. The portions 1814 and 1816 may be combined (as indicated by reference numeral 1818) upstream of the cooler heat exchanger 128.

The addition of the two parallel cross-exchangers 1802 and 1804 provided for cooling the effluent and therefore reduce cooling water demand for cooling the effluent 104 (e.g., reduce demand of cooling tower water at the cooler heat exchanger 128) as compared to Option 1. Furthermore, the addition of the two parallel cross-exchangers 1802 and 1804 recover heat from the effluent 104 for feed saturation (e.g., for saturation of the ethane gas 112 and the mixed feed 108 with water). Therefore, steam consumption (e.g., LP steam at the circulation water heater 118) for feed saturation may be reduced as compared with Option 1. However, the addition of the two parallel cross-exchangers 1802 and 1804 between the ODH reactor 102 and PGC 158 may result in a lower suction pressure for PGC 158 and thus higher PGC 158 power consumption as compared to Option 1 (FIG. 1).

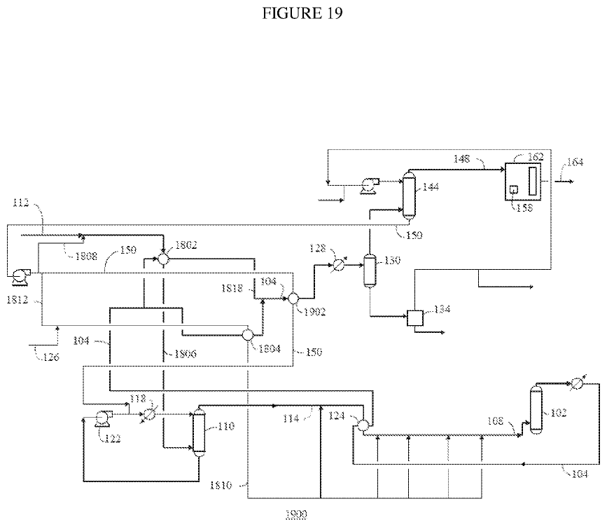

FIG. 19 is an ethylene production system 1900 that may be labeled as Option 18. The ethylene production system 1900 is the same or similar as the ethylene production system 1800 of FIG. 18 but with the addition of a recycle water cross-exchanger 1902. The recycle water cross-exchanger 1902 heats (with effluent 104 as heating medium) the recycle water 150 in route to the ethane saturator tower 110. This may further reduce steam consumption (e.g., LP steam at the circulation water heater 118) for feed saturation as compared with Option 17 (FIG. 18). The addition of the recycle water cross-exchanger 1902 may also further reduced cooling water demand at the cooler heat exchanger 128 for cooling the effluent 104 as compared to Option 17. However, the addition of another heat exchanger (recycle water cross-exchanger 1902) in which the effluent 104 may add extra pressure drop between ODH reactor 102 and PGC 158, which could lead to more power demand by the PGC 158.

In the illustrated embodiment, the recycle water cross-exchanger 1902 is operationally disposed along the effluent 104 flow downstream of the cross-exchangers 1802 and 1804 and upstream of the cooler heat exchanger 128.

The recycle water 150 is the bottom streams discharged from the acetic acid scrubber 144. Portions 1808 and 1812 of the recycle water 150 are taken for partially saturating the ethane gas 112 and oxygen gas 126, as is done in FIG. 18. However, the remaining recycle water 150 is routed through the recycle water cross-exchanger 1902 before being sent through the circulation water heater 118 to the ethane saturator tower 110.

As with cross-exchangers 1802 and 1804, the recycle water cross-exchanger 1902 may be a shell-and-tube heat exchanger, plate heat exchanger, or a plate-fin heat exchanger, or other type of heat exchanger. Further, as generally for the cross-exchangers discussed herein, the system 1900 may be configured for routing the heating medium and cooling medium through either side of the cross-exchanger, respectively. For instance, the cross-exchanger as a shell-and-tube heat exchanger may be configured such that the heating medium (effluent 104 for cross-exchanger 1902) flows through the tubes (tube bundle) and the cooling medium (recycle water 150 for cross-exchanger 1902) flows through the shell. Alternatively, the cross-exchanger may be configured such that the heating medium flows through the tubes and the cooling medium flows through the shell.

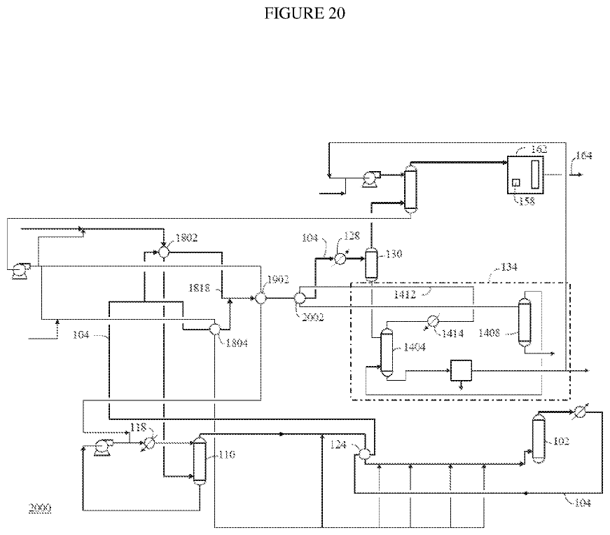

FIG. 20 is an ethylene production system 2000 that may be labeled as Option 19. The ethylene production system 2000 is the same or similar as the ethylene production system 1900 of FIG. 19 but with the addition of an extract cross-exchanger 2002. The extract cross-exchanger 2002 heats (with effluent 104 as heating medium) the extract 1412 of the extractor column 1404. This may reduce steam demand (e.g., LP steam) at the reboiler of the solvent recovery column 1408 in the acetic acid unit 134 as compared to Option 18 (FIG. 19). The addition of the extract cross-exchanger 2002 may also further reduced cooling water demand at the cooler heat exchanger 128 for cooling the effluent 104 as compared to Option 18. However, the addition of another heat exchanger (extract cross-exchanger 2002) in which the effluent 104 is routed may add extra pressure drop between ODH reactor 102 and PGC 158, which could lead to more power demand by the PGC 158.

In the illustrated embodiment, the extract cross-exchanger 2002 is operationally disposed along the effluent 104 flow between the recycle heat exchanger 1902 and the cooler heat exchanger 128. The extract 1412 can be vaporized (partially or completely) in the extract cross-exchanger 2002 and the vapor may be superheated in the extract cross-exchanger 2002.

As with cross-exchangers previously discussed, the extract cross-exchanger 1902 may be a shell-and-tube heat exchanger, plate heat exchanger, or a plate-fin heat exchanger, or other type of heat exchanger. Further, as also discussed generally for cross-exchangers, the heat source (effluent 104) and heat sink (extract 1412) may be on either side.

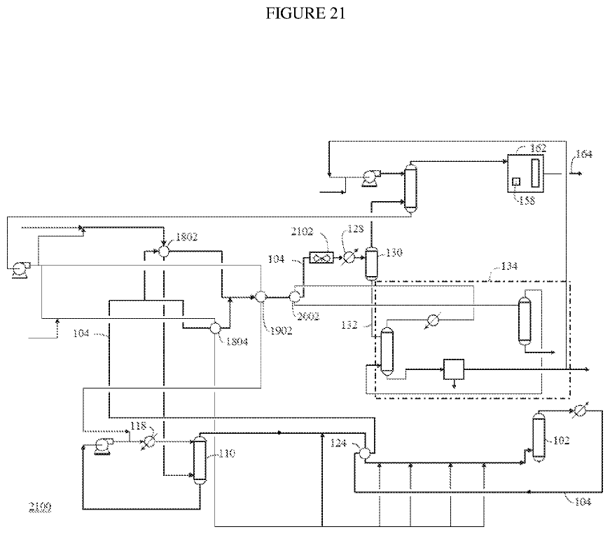

FIG. 21 is an ethylene production system 2100 that may be labeled as Option 20. The ethylene production system 2100 is the same or similar as the ethylene production system 2000 of FIG. 20 (Option 19) but with the addition of an air cooler 2102 to cool the effluent 104. The air cooler 2102 is operationally disposed between the extract cross-exchanger 2002 and the cooler heat exchanger 128 to cool the effluent 104. This may reduce the cooling water (e.g., cooling tower water) demand by the cooler heat exchanger 128 as compared to Option 19 (FIG. 20). However, adding another heat exchanger (air cooler 2102) along the effluent 104 flow may add pressure drop between reactor 102 and PGC 158 and thus could lead to more power demand by PGC 158.

The air cooler 2102 may be similar to the aforementioned air coolers. The air cooler 2102 is a heat exchanger that may be fan heat exchanger including one or more fans and that can include fins or a finned-tube bundle. The air cooler 202 may be a fin-fan heat exchanger. The cooling medium may be ambient air.

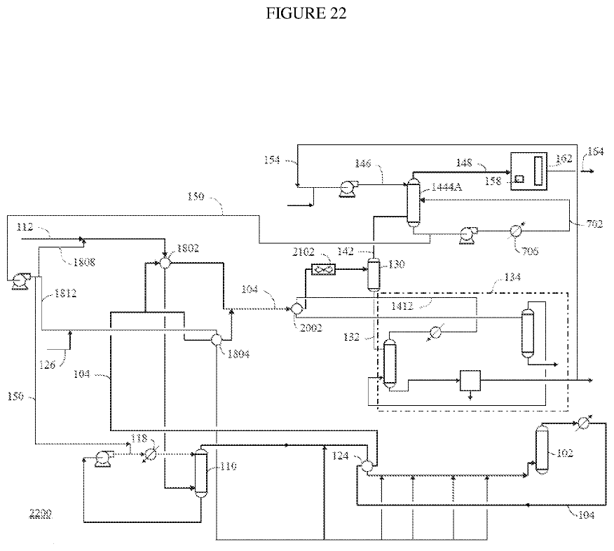

FIG. 22 is an ethylene production system 2200 that may be labeled as Option 21. The ethylene production system 2200 is the same or similar as the ethylene production system 2100 of FIG. 21 (Option 20) but without the cooler heat exchanger 128 and the recycle water cross-exchanger 1902, and with the acetic acid scrubber 144 instead as quench/acetic acid scrubber 144A (e.g., see previous figures). This may concentrate acetic acid in raw acetic acid 132 (and thus reduce the heating and cooling requirement in the acetic acid unit 134) as compared to Option 20 (FIG. 21). It may also increase the temperature of recycle water 150 back to ethane saturator tower 110 (and leading to elimination of the recycle water cross-exchanger 1902 for the recycle water 150 against reactor effluent) as compared to Option 20. Lastly, because two heat exchangers (cooler heat exchanger 128 and recycle water cross-exchanger 1902) are eliminated from ODH reactor 102 effluent 104 side, the pressure drops between the ODH reactor 102 and PGC 158 may be reduced, which could lead to less PGC 158 power demand as compared to Option 20 (FIG. 21).

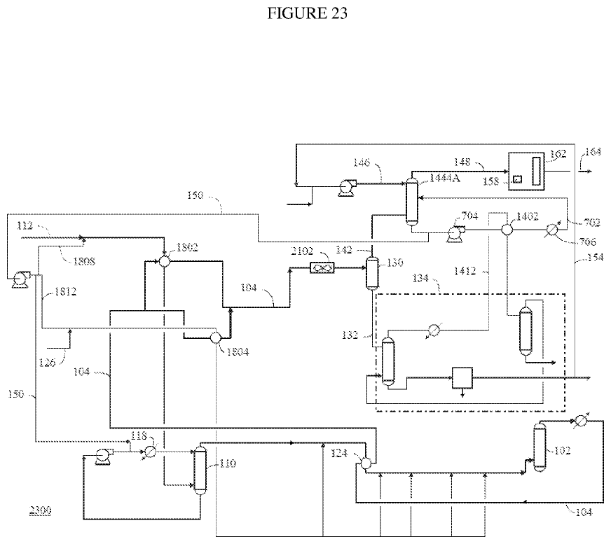

FIG. 23 is an ethylene production system 2300 that may be labeled as Option 22. The ethylene production system 2300 is the same or similar as the ethylene production system 2200 of FIG. 22 (Option 21) but without the extract cross-exchanger 2002 against effluent 104 and with the extract cross-exchanger 1402 (e.g., see FIG. 14) against quench water 702 to heat the extract 1412. The extract 1412 can be partially vaporized or fully vaporized (and the vapor may be superheated) in the extract cross-exchanger 1402.

The heat load for heating the extract 1412 is shifted from the effluent 104 to the quench water 702. Thus, the heat removed from the effluent 104 by the extract cross-exchanger 2002 in FIG. 22 (Option 21) is shifted to the air cooler 2102 in FIG. 23 (Option 22). This could mean that the air cooler 2102 in Option 22 will be larger (more cooling capability) than in Option 21. However, because heat from the quench water 702 is removed for the extract 1412 via the extract heat exchanger 1402 in Option 22, the quench-water cooler 706 may beneficially be smaller (less cooling water demand) in Option 22 as compared to Option 21. Furthermore, because the extract cross-exchanger 2002 along the effluent 104 flow is eliminated in Option 22, the pressure drop between ODH reactor 102 and PGC 158 may be less, which could lead to less PGC 158 power demand as compared to Option 21.

Options 1-22 may be presented with respect to each other and can encompass incremental differences with respect to each other. For a description of text, designations, and reference numerals depicted in a given figure of FIGS. 1-23, see also the discussion of the other figures of FIGS. 1-23.

As can be appreciated, the vessels and heat exchangers discussed with respect to FIGS. 1-23 may have at least one inlet (e.g., nozzle) that is a flanged or screwed connection with an inlet conduit, at least one outlet (e.g., nozzle) that is a flanged or screwed connection with an outlet conduit.

More than one ODH reactor 102 may be employed, including in series and/or parallel. Although the ODH reactor 102 is depicted as a or one-stage reactor, e.g., with all the feed components (mixed feed 108) added at the inlet of the reactor, the processes described are applicable for other reactor configurations, including multiple stage reactors and reactors with multiple inter-stage feed additions.

The steam generated or utilized may be low pressure (LP) steam (e.g., 150 psig or less), medium pressure (MP) steam (e.g., in the range of 150 psig to 600 psig), high pressure (HP) steam (e.g., 600 psig or greater), or very high pressure (VHP) steam (e.g., 1500 psig or greater), and so forth. Again, at the steam generation heat exchanger 106, generation of HP steam or VHP steam may be generally be more valuable than generating MP steam or LP steam and thus improve economics of the ethylene production system 100. There may be different applications for the steam. The use of the steam by the consumers or customers receiving the steam may depend on the pressure or quality of the steam. In some implementations, higher steam pressures of the produced steam may give more versatility in the integration of the steam within the facility or plant. For instance, HP steam can be utilized to power turbines attached to compressors, while LP steam is typically used for heating purposes, and the like.

As indicated, the ODH reactor 102 may be a fixed-bed reactor (e.g., a tubular fixed-bed reactor), a fluidized-bed reactor, an ebullated bed reactor, or a heat-exchanger type reactor, and so on. A fixed-bed reactor may have a cylindrical tube(s) filled with catalyst pellets as a bed of catalyst. In operation, reactants flow through the bed and are converted into products. The catalyst in the reactor may be one large bed, several horizontal beds, several parallel packed tubes, or multiple beds in their own shells, and so on.

A fluidized bed reactor may be a vessel in which a fluid is passed through a solid granular catalyst (e.g., shaped as spheres or particles) at adequate velocity to suspend the solid catalyst and cause the solid catalyst to behave as though a fluid. In implementations, a fluidized bed reactor may have a support for the catalyst. The support may be a porous structure or distributor plate and disposed in a bottom portion of the reactor. Reactants may flow upward through the support at a velocity to fluidize the bed of catalyst (e.g., the catalyst rises and begins to swirl around in a fluidized manner). A fluidized bed reactor has a recirculating mode of operation.

The techniques may include maintaining an operating temperature of the ODH reactor 102 at less than 450° C., less than 425° C., or less than 400° C. As for operating pressure, the ODH reactor 102 inlet pressure may be less than 80 pound per square inch gauge (psig), or less than 70 psig. The reactor inlet pressure for each reactor may be in the range of 1 psig to 80 psig, or in the range of 5 psig to 75 psig. Other operating conditions of the ODH reactor 102 in the embodiments of the ODH reactor 102 as a tubular fixed-bed reactor may be gas hourly space velocity (GHSV) in the range of 200 hour⁻¹ to 40,000 hour⁻¹.

Options 1-22 can generally be compared for energy integration of ODH reactor effluent cooling and acetic acid recovery, and with consideration of ODH reactor feed saturation. Option 1 is utilized as a base case for the comparison. In other words, Options 2-22 may be compared to Option 1 as a baseline case. The second column in Tables 1 and 2 give a “Comparison Base” for equipment and operation.

Based on the energy integration, the example Options 1-22 in are evaluated for the section of the facility process from receiving/processing the reactor feed through the first stage of the PGC 158. In certain implementations for that section of the process, various options of example Options 1-22 can reduce steam consumption by up to 51%, power demand by up to 30%, and cooling water demand by up to 76% while concentrating raw acetic acid 132 to the acetic acid unit 134 by up to 67%. Such may result in not only overall lower operating expense of the ethylene production system up to the PGC 158 but also lower capital expense for the acetic acid unit 134, cooling water system, and steam system. However, the present techniques are not limited to these numerical values.

Process simulations were performed with Aspen Plus® V10. The SR-POLAR equation of state was utilized for the simulations. For the simulations, the feed inlet temperature (mixed feed 108) into the ODH reactor 102 is maintained below 310° C. at 465 kilopascal (kPa) and oxygen concentration in the mixed feed 108 (MIXED-FD) into the ODH reactor 102 is targeted at 10 volume percent (vol %) in order to be outside the flammability zone. The oxygen to ethane molar ratio in the mixed feed 108 stream is 0.62. Total water content to ODH reactor 102 is 74 vol % which requires heating to evaporate the water before the ODH reactor 102 and cooling to condense the water after the ODH reactor 102. Tables 1 and 2 show the impact on heating, cooling, and power (water circulation pump of feed saturator, CW system pumping and fans, air cooler fans, 1st stage PGC 158) of all presented Options 2-22 (FIGS. 2-23) relative to Option 1 (FIG. 1) based on different reactor effluent cooling strategies and other energy integration aspects. The example results show the following. Other aspects of the present techniques fall outside of these example results.

Option 1 requires significant amount of steam for feed saturation and acetic acid (AA) unit, significant amount of cooling water for reactor effluent cooling and AA unit, and much power for feed saturation (with water) and for the cooling water (CW) system that includes a cooling tower. The feed saturation generally refers to ethane saturation via the ethane saturator ethane saturator tower 110 but can involve partial saturation of ethane and oxygen via heat exchangers, and ultimately to give saturation of the mixed feed 108 to the ODH reactor 102.

Option 2 requires significantly less cooling water but more air cooling. The overall power consumption would be slightly less than Option 1.

Option 3 has much higher concentration of AA in the raw AA concentration with much less flowrate of the raw AA concentration. Thus, the AA unit would be much smaller with significantly less heating and cooling demand. Due to higher temperature of recycle water to feed saturator, the steam consumption is lower at feed saturator compared to option 1 and consequently less cooling water is required to cool down the reactor effluent.

Option 4 is similar to Option 3 but with significantly less CW demand for cooling of the ODH reactor effluent cooling and a big air cooler for cooling the effluent.

Option 5 is similar to Option 4 but with significantly less CW demand for the ODH reactor effluent cooling and another big air cooler for cooling the effluent.

Option 6 is similar to Option 2 but with slightly higher acetic-acid concentration in the raw AA and lower total flowrate of the raw AA, which would result in lower heating but higher cooling demand at the AA unit. Because the AA scrubber delivers higher recycle water temperature to the feed saturator, the feed saturator has slightly lower steam consumption. The overall power consumption is much lower than Option 2.

Option 7 is similar to Option 6 but with much higher acetic-acid concentration in the raw AA and much lower total flowrate of the raw AA, which would result in significantly lower heating and cooling demand at AA unit. Because the AA scrubber delivers higher recycle water temperature to the feed saturator, the feed saturator has lower steam consumption as compared to Option 6. The overall power consumption is much lower than Option 6.

Option 8 has same acetic-acid concentration in the raw AA concentration and the same utility demand for the AA unit as Option 5. Because the AA scrubber delivers higher recycle water temperature to the feed saturator, the feed saturator has lower steam consumption than Option 5. The overall power consumption is much lower than Option 5. Consequently, Option 5 requires less CW demand for reactor effluent cooling than Option 5. The greatest impact would be on overall power consumption which would significantly be reduced compared to Option 5.

Option 9 is similar to Option 8 but with much more CW demand for reactor effluent cooling. Because the quench/scrubber tower delivers higher recycle water temperature to the feed saturator, the feed saturator has lower steam consumption than Option 8. The overall power consumption is lower than Option 8 due to less pressure drop in Option 9 on reactor effluent side and thus less power at PGC.

Option 10 has same AA concentration in the raw AA and the same utility demand for AA unit as Option 9. Option 10 requires significantly more CW for reactor effluent cooling as compared to Option 9. However, the overall power consumption is lower than Option 9 due to less pressure drop between the ODH reactor and the PGC.

Option 11 is similar to Option 10 but with significantly less AA concentration in the raw AA and much higher flowrate of raw AA, which would result in significantly more heating and cooling demand in the AA unit. Also, the quench/scrubber delivers lower recycle water temperature to the feed saturator, which would result in higher steam consumption at the feed saturation system as compared to Option 10. The overall power consumption would be significantly higher than Option 10.

Option 12 is similar to Option 11 but with slightly higher recycle water temperature to feed saturator, which would result on slightly lower steam demand at the feed saturation system.

Option 13 is similar to Option 9 but with high heat recovery for the AA unit from reactor effluent cooling system, which would result in significantly less heat demand for the AA unit and significantly less CW demand for the reactor effluent cooling. The overall power consumption would be lower than Option 9.

Option 14 is similar to Option 10 but high heat recovery for the AA unit from reactor effluent cooling system, which would result in significantly less heat demand for the AA unit and significantly less CW demand for the reactor effluent cooling. The overall power consumption would be much lower than Option 10.

Option 15 is similar to Option 14 but with off-loading cooling water system using air coolers. Option 15 has slightly lower power consumption than Option 14.

Option 16 is similar to Option 11 but with high heat recovery for the AA unit from reactor effluent cooling system, which would result in significantly less heat demand for the AA unit and significantly less CW demand for reactor effluent cooling. Option 16 would also recover a small amount of heat for ethane and oxygen preheating from reactor effluent cooling system, which would have small impact on lowering steam consumption of the feed saturator. The overall power consumption would be much lower than Option 11.

Option 17 is similar to Option 1 with significant heat recovery from the reactor effluent to partially saturate ethane and oxygen feed, which would drastically reduce steam demand for feed saturation and also for CW demand for reactor effluent cooling. The overall power consumption would be significantly lower than Option 1.

Option 18 is similar to option 17 with more heat recovery from reactor effluent to preheat the recycle water back to feed saturator which would result on further reduction of steam demand for feed saturation and CW demand for reactor effluent cooling.

Option 19 is similar to Option 18 but with significantly more heat recovery from reactor effluent to vaporize (including partially vaporize or superheat vapor) the “AA extract” from “AA extractor” to the solvent recovery tower. This would significantly reduce the heating demand at AA unit while drastically reducing CW demand for reactor effluent cooling compared to Option 18. The overall power consumption would be significantly lower than Option 18.

Option 20 is similar to Option 19 with significantly less CW demand for the reactor effluent cooling while adding a big air cooler. The overall power consumption would be higher than Option 19.

Option 21 is the combination of Option 8 and Option 20. The AA concentration in the raw AA is significantly higher and the flowrate of the raw AA significantly lower, which would result in significantly less heating and cooling demand at the AA unit as compared to Option 20. Due to heat recovery from reactor effluent for “AA extract” heating/vaporization, Option 21 requires significantly less heating for the AA unit as compared to Option 8. Due to heat recovery for feed saturation and delivering much higher recycle water temperature to the feed (ethane) saturator, the steam demand for feed saturation would be less than in Option 8 and Option 20. However, CW demand for reactor effluent cooling would be much higher than Option 20 but with a much smaller air cooler in Option 21. The overall power consumption would be significantly lower than Option 20.

Option 22 is the combination of Option 13 and Option 21. The “AA extract” would be vaporized against quench water. The air cooler for reactor effluent is larger than Option 21 but the quench water cooler is much smaller. The overall power consumption would be lower than Option 21.

Results of comparison calculations based on the process simulations are given in Table 1 and Table 2. The comparison base is a logical contrast for the given option. The results of Options 2-22 in Table 1 and Table 2 are given as a change (in percent) relative to Option 1. Table 1 gives the relative percent comparison for the AA concentration in the raw AA, the total mass flow rate of raw AA, the LP steam consumption for feed saturation (e.g., at the ethane saturator), steam consumption at the AA unit, and total heat (steam consumption) for combination of feed saturation and the AA unit. Table 2 gives the relative percent comparison for the CW demand for reactor effluent cooling, the air cooling demand for the reactor effluent cooling, CW demand for the AA unit cooling, total CW demand for the combination of the reactor effluent cooling and the AA unit cooling, and the power demand by the combination of the Saturator, 1^{st} stage compressor of PGC, CW system, and air cooler(s).

TABLE 1
Comparison of Options

| | | | | | Acetic | Total Heat for |
|---|---|---|---|---|---|---|
| | | | | FEED | Acid | FD SAT & |
| | | Raw Acetic Acid | | Saturation | Unit | AA Unit |
| | | AA Conc. | Total Mass | LP Steam | Steam | Steam |
| | | Change | Flowrate | Consumption | Consumption | Consumption |
| | | Relative to | Relative to | Relative to | Relative to | Relative to |
| | Comparison | Option 1 | Option 1 | Option 1 | Option 1 | Option 1 |
| Options | Base | (%) | (%) | (%) | (%) | (%) |
| Option 1 | — | 100% | 100% | 100% | 100% | 100% |
| Option 2 | Option 1 | 100% | 100% | 100% | 99% | 100% |
| Option 3 | Option 1 | 167% | 54% | 94% | 61% | 83% |
| Option 4 | Option 2/3 | 167% | 54% | 94% | 61% | 83% |
| Option 5 | Option 4 | 167% | 54% | 94% | 61% | 83% |
| Option 6 | Option 2 | 111% | 88% | 92% | 94% | 93% |
| Option 7 | Option 6 | 167% | 54% | 85% | 61% | 77% |
| Option 8 | Option 5 | 167% | 54% | 90% | 61% | 80% |
| Option 9 | Option 8 | 167% | 54% | 85% | 61% | 77% |
| Option 10 | Option 9 | 167% | 54% | 85% | 61% | 77% |
| Option 11 | Option 1/10 | 88% | 111% | 93% | 98% | 95% |
| Option 12 | Option 11 | 88% | 111% | 91% | 98% | 94% |
| Option 13 | Option 9 | 167% | 54% | 85% | 28% | 66% |
| Option 14 | Option 10 | 167% | 54% | 85% | 28% | 66% |
| Option 15 | Option 14 | 167% | 54% | 85% | 28% | 66% |
| Option 16 | Option 11 | 88% | 111% | 92% | 48% | 77% |
| Option 17 | Option 1 | 100% | 100% | 70% | 100% | 80% |
| Option 18 | Option 17 | 100% | 100% | 63% | 100% | 75% |
| Option 19 | Option 18 | 98% | 99% | 62% | 48% | 57% |
| Option 20 | Option 19 | 104% | 93% | 62% | 48% | 57% |
| Option 21 | Option 8/20 | 165% | 54% | 60% | 28% | 49% |
| Option 22 | Option 21 | 165% | 54% | 60% | 28% | 49% |

TABLE 2
Comparison of Options

| | | | | | | Power by |
|---|---|---|---|---|---|---|
| | | | | | Total CW | Saturator, |
| | | Reactor Effluent | | AA Unit | for Rx Eff | PGC, |
| | | Cooling Demand | | Cooling | Cooling & | CW System, |
| | | | Air Cooling | Demand | AA Unit | Air Cooler |
| | | CW | Demand | CW | CW | Power |
| | | Demand | Relative to | Demand | Demand | Demand |
| | | Relative to | CW Demand of | Relative to | Relative to | Relative to |
| | Comparison | Option 1 | Option 1 | Option 1 | Option 1 | Option 1 |
| Options | Base | (%) | (%) | (%) | (%) | (%) |
| Option 1 | — | 100% | 0% | 100% | 100% | 100% |
| Option 2 | Option 1 | 11% | 89% | 98% | 36% | 98% |
| Option 3 | Option 1 | 90% | 0% | 75% | 85% | 101% |
| Option 4 | Option 2/3 | 40% | 49% | 75% | 50% | 100% |
| Option 5 | Option 4 | 9% | 81% | 75% | 28% | 103% |
| Option 6 | Option 2 | 5% | 81% | 111% | 36% | 92% |
| Option 7 | Option 6 | 32% | 49% | 75% | 44% | 87% |
| Option 8 | Option 5 | 4% | 81% | 75% | 24% | 93% |
| Option 9 | Option 8 | 32% | 49% | 75% | 44% | 88% |
| Option 10 | Option 9 | 79% | 0% | 75% | 78% | 85% |
| Option 11 | Option 1/10 | 79% | 0% | 132% | 94% | 94% |
| Option 12 | Option 11 | 78% | 0% | 132% | 94% | 94% |
| Option 13 | Option 9 | 17% | 49% | 80% | 35% | 83% |
| Option 14 | Option 10 | 64% | 0% | 80% | 69% | 79% |
| Option 15 | Option 14 | 43% | 21% | 80% | 54% | 78% |
| Option 16 | Option 11 | 53% | 0% | 147% | 80% | 85% |
| Option 17 | Option 1 | 77% | 0% | 100% | 83% | 84% |
| Option 18 | Option 17 | 71% | 0% | 100% | 79% | 84% |
| Option 19 | Option 18 | 47% | 0% | 106% | 64% | 79% |
| Option 20 | Option 19 | 12% | 35% | 106% | 39% | 83% |
| Option 21 | Option 8/20 | 32% | 15% | 79% | 45% | 75% |
| Option 22 | Option 21 | 17% | 30% | 79% | 35% | 70% |

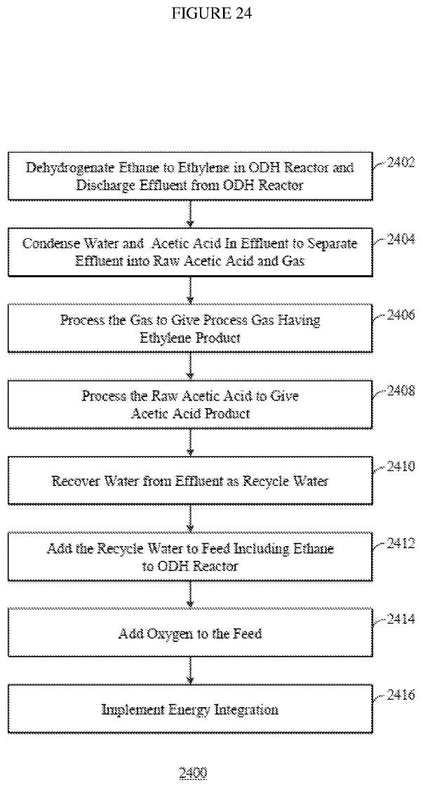

FIG. 24 is a method 2400 of producing ethylene. At block 2402, the method includes dehydrogenating ethane to ethylene via an ODH catalyst in presence of oxygen in an ODH reactor. The ODH reactor may be, for example, a fixed bed reactor or a fluidized bed reactor. Acetic acid may be produced in the ODH reactor as a byproduct of the ODH reaction that dehydrogenates the ethane to ethylene. The method includes discharging an effluent from the ODH reactor. The effluent includes ethylene, water, acetic acid, carbon dioxide, carbon monoxide, and unreacted ethane. In some implementations, heat from the effluent can be used to heat water (e.g., boiler feedwater) in a heat exchanger to generate steam for consumption at the facility having the ODH reactor.

At block 2404, the method includes condensing water and acetic acid in the effluent to separate the effluent into liquid raw acetic acid and gas. To condense the water and acetic acid, the effluent may be cooled in a heat exchanger (e.g., with cooling water, air, etc.) and also in a quench tower in some implementations. The raw acetic acid includes the condensed water and the condensed acetic acid. The raw acetic acid is typically primarily water (greater than 50 wt %). The concentration of acetic acid in the raw acetic acid can be less than 1 wt % in some implementations. The gas is the remainder of the effluent, which is ethylene, carbon dioxide, carbon monoxide, and unreacted ethane. The gas can include a relatively small amount of acetic acid and water. The separation of the raw acetic acid from the gas can occur, for example, in a flash drum or a quench tower.

At block 2406, the method includes processing the separated gas to give process gas having ethylene product. The processing may include to remove the small amount of acetic acid and water in the gas, such as via scrubbing or quenching in one or more towers. The discharged process gas includes ethylene, ethane, carbon dioxide, and carbon monoxide. The process gas may have, for example, less than 50 ppmv of acetic acid and less than 5 mol % of water vapor. The amount of ethylene in the process gas may be, for example, in the range of 10 mol % to 90 mol %. The process gas may be forwarded to a process gas compressor and further processed to recover the ethylene product.

At block 2408, the method includes processing the raw acetic acid, such as in an acetic acid unit, to give acetic acid product. The acetic acid unit may include, for example, an extractor column injection of solvent to remove acetic acid from the raw acetic acid, a water stripper tower to process raffinate from the extractor column to recover water, and a solvent recovery column (vessel) to remove the solvent from the extract (primarily) acetic acid discharged from the extractor column to give the acetic acid product. See, for example, the discussion of the acetic acid unit 134 for FIG. 14.

At block 2410, the method includes recovering water from the effluent as recycle water for feed dilution. The processing of the raw acetic acid to give the acetic acid product can give water as recycle water. Thus, the processing (block 2408) of the raw acetic acid can include recovering the water as recycle water in block 2410. For instance, the water stripper tower in the acetic acid unit can give water both for recycle and as water product.

For a water system for diluting the reactor feed that is substantially closed-circuit, the amount of water product (e.g., from the water stripper tower in the acetic acid unit) may be approximately the amount of water generated in the ODH reactor.

In implementations, the recycle water can flow from the acetic acid unit as scrubbing liquid to a tower processing (e.g., scrubbing) the aforementioned separated gas (block 2406). Recycle water for feed dilution can be taken as a bottoms stream (or from a higher section) of the tower. Therefore, the processing (block 2408) of the raw acetic acid to give acetic acid product in combination with the processing (block 2406) of the separated gas to give the process gas can provide for recovering (block 2410) the water from the effluent as recycle water.

Thus, the processing of the raw acetic acid may include the recovering water as recycle water. In other words, the processing of the raw acetic acid may provide for the action of recovering water from the effluent as recycle water. Moreover, the processing of the raw acetic acid to give acetic acid product and the processing the gas to give the process gas in combination may provide for the recovering the water from the effluent as recycle water.

At block 2412, the method includes adding the recycle water to the feed including ethane in route to the ODH reactor. The recycle water may added to the ethane in an ethane saturator tower. The recycle water may be added to the ethane in a conduit conveying the ethane. The recycle water may be added to oxygen (in a conduit) that is added to the feed having the ethane. The method may include providing the feed including ethane to the ODH reactor. The ethane may be ethane gas provided from a supply pipeline, or can be ethane liquid provided from a supply pipeline and that is vaporized to ethane gas. As used herein, the term ethane is generally meant to be ethane gas.

At block 2414, the method includes adding oxygen (O₂ gas) to the feed including the ethane to give a mixed feed to the ODH reactor. As used herein, the term oxygen is generally meant to be O₂ gas. The oxygen may be added at a single addition point to a conduit conveying the feed including the ethane or at multiple addition points to the conduit conveying the feed including the ethane. In some implementations, as mentioned, water (e.g., recycle water) may be added to the oxygen for feed dilution with the water prior to addition of the oxygen to the feed. The mixed feed to the reactor includes the ethane gas and the oxygen gas. The mixed feed may include recycle water added (block 2412) for the feed dilution.

At block 2416, the method includes implementing energy integration in the ODH reactor system including with respect the processing of the effluent. For instance, the method can include heating the feed having the ethane to the ODH reactor with the effluent, such as in a cross-exchanger. This may cool the effluent and therefore contribute to cooling the effluent for condensing acetic acid and water in the effluent. The method may include providing heat from the effluent to processing the raw acetic acid. For example, the effluent may be utilized to heat (e.g., in a cross-exchanger) the extract discharged from the extractor column in the acetic acid unit. Such may cool the effluent and thereby contribute to cooling the effluent for condensing water and acetic acid in the effluent.

The processing of the gas to give the process gas may provide heat to processing the raw acetic acid. For example, quench water in a circulating quench water loop (for a tower quenching the gas) may heat (e.g., in a cross-exchanger) the extract discharged from the extractor column. The processing of the gas to give the process gas may heat ethane provided for the feed including the ethane. For example, quench water in a circulating quench water loop (for a tower quenching the gas) may heat (e.g., in a cross-exchanger) ethane from a supply pipeline before recycle water is added to the ethane. The processing of the gas to give the process gas may heat the recycle water. For example, quench water in a circulating quench water loop (for a tower quenching the gas) may heat (e.g., in a cross-exchanger) the recycle water. Such may beneficially contribute to the heating for feed dilution.

The method may include heating (e.g., in a cross-exchanger) the recycle water with the effluent, thereby cooling the effluent (and thus contribute to the cooling load to condense water and acetic acid in the effluent). As indicated, the method may include heating the feed including the ethane and recycle water with the effluent, and thereby cooling the effluent (and thus contribute to the cooling load to condense water and acetic acid in the effluent).

The method may include heating (e.g., in a cross-exchanger) the ethane (provided for the feed) with the effluent. The method may include heating (e.g., in a cross-exchanger) the oxygen with the effluent. In one example, a mixture of ethane and recycle water is heated in the cross-exchanger with the effluent for the feed. In another example, a mixture of oxygen and recycle water is heated with the effluent in the cross-exchanger (e.g., for partial saturation of the oxygen with water) prior to addition to the feed having the ethane. The heating of ethane, oxygen, or mixtures including recycle water may cool the effluent (which may contribute to the cooling load to condense water and acetic acid in the effluent).

An embodiment is a method of producing ethylene, including dehydrogenating ethane to ethylene via an ODH catalyst in presence of oxygen in an ODH reactor, thereby forming acetic acid in the ODH reactor, and discharging an effluent including at least ethylene, acetic acid, and water from the ODH reactor through a steam-generation heat exchanger to generate steam with heat from the effluent, thereby cooling the effluent. The method includes flowing the effluent from the steam-generation heat exchanger through a feed heat exchanger to heat a feed having ethane for the ODH reactor with the effluent, thereby cooling the effluent. The method includes recovering acetic acid from the effluent as acetic acid product and forwarding a process gas having ethylene from the effluent for further processing to give ethylene product. The method may include further cooling the effluent downstream of the feed heat exchanger, thereby condensing water in the effluent. The further cooling the effluent downstream of the feed heat exchanger may include cooling the effluent with at least one of a cooler heat exchanger or an air cooler, wherein the cooler heat exchanger utilizes cooling water as a cooling medium, and wherein the air cooler is a fan heat exchanger that utilizes air as a cooling medium. The further cooling of the effluent downstream of the feed heat exchanger may include cooling the effluent in a quench tower or in an acid scrubber having a quenching section. The method may include further cooling the effluent downstream of the feed heat exchanger to separate the effluent as further cooled and having the water as condensed into raw acetic acid and gas, wherein the gas includes ethylene, water, acetic acid, ethane, carbon dioxide, and carbon monoxide, and wherein the raw acetic acid comprises acetic acid and water. The further cooling the effluent downstream of the feed heat exchanger and separating the effluent as further cooled into gas and raw acetic acid may involve processing the effluent in a quench tower or in an acetic acid scrubber having a quenching section, and wherein the method includes discharging the raw acetic acid from a bottom portion of the quench tower or from a bottom portion of the acetic acid scrubber having the quenching section. The further cooling the effluent downstream of the feed heat exchanger may include cooling the effluent in a heat exchanger, wherein separating the effluent includes separating the effluent in a flash drum into the gas and the raw acetic acid, and wherein the method includes discharging the gas overhead from the flash drum and discharging the raw acetic acid from a bottom portion of the flash drum.

The method may include removing water and acetic acid from the gas to give the process gas including the ethylene, ethane, carbon dioxide, and carbon monoxide, wherein the process gas has less than 50 ppmv of acetic acid and less than 5 mol % of water vapor. The method may include providing water and acetic acid removed from the gas as recycle water for saturating the feed including ethane with water. The method may include heating at least a portion of the recycle water in a cross-exchanger with the effluent as a heating medium. The method may include combining at least a portion of the recycle water with oxygen gas to give a mixture, heating the mixture in a cross-exchanger with effluent as a heating medium, and adding the mixture as heated to the feed including the ethane. The forwarding the process gas for further processing may involve forwarding the process gas to a process gas compressor. The method may include providing the raw acetic acid to an acetic acid unit having an extractor column that is a liquid-liquid extraction column, wherein recovering acetic acid from the effluent as acetic acid product includes processing the raw acetic acid in the acetic acid unit.

The processing the raw acetic acid in the acetic acid unit may include providing the raw acetic acid and solvent to the extractor column, discharging extract (primarily acetic acid) overhead from the extractor column, and heating the extract in a cross-exchanger with a heating medium, wherein the heating medium includes the effluent downstream of the feed heat exchanger, or wherein the heating medium includes quench water. The extract includes the solvent and a relatively small amount of water, wherein the extract includes more of the solvent than water. The method may include discharging the acetic acid product having at least 99 wt % acetic acid from the acetic acid unit, wherein the raw acetic acid has a concentration of acetic acid in a range of 0.3 wt % to 45 wt %, and wherein the acetic acid unit includes a solvent recovery column that is a distillation column.

Another embodiment is a method of producing ethylene, including dehydrogenating ethane to ethylene via an ODH catalyst in presence of oxygen in an ODH reactor, thereby forming acetic acid in the ODH reactor, and discharging an effluent including ethylene, acetic acid, water, carbon monoxide, carbon dioxide, and unreacted ethane from the ODH reactor through a steam-generation heat exchanger to generate steam, wherein the steam-generation heat exchanger transfers heat from the effluent to water to generate the steam, thereby cooling the effluent. The method includes flowing the effluent from the steam-generation heat exchanger through a feed heat exchanger to heat a feed for the ODH reactor with the effluent, wherein the feed heat exchanger transfers heat from the effluent to the feed, thereby cooling the effluent. The method includes cooling the effluent downstream of the feed heat exchanger, thereby condensing water in the effluent. The method includes forwarding process gas having ethylene from the effluent to a process gas compressor for further processing to give ethylene product. The cooling of the effluent downstream of the feed heat exchanger may include cooling the effluent with at least one of a cooler heat exchanger or an air cooler, wherein the cooler heat exchanger utilizes cooling water as a cooling medium, and wherein the air cooler is a fan heat exchanger that utilizes air as a cooling medium.

The method may include providing the effluent having the condensed water to a flash drum and separating in the flash drum the effluent into gas and raw acetic acid, wherein the gas comprises ethylene, water, acetic acid, ethane, carbon dioxide, and carbon monoxide, wherein the raw acetic acid includes acetic acid and water. The method may include discharging the raw acetic acid from a bottom portion of the flash drum to an acetic acid unit having an extractor column that is a liquid-liquid extraction column and a solvent recovery column that is a distillation column, and processing the raw acetic acid in the acetic acid unit to give acetic acid product. The method may include discharging the gas overhead from the flash drum, and removing water and acetic acid from the gas to give the process gas including the ethylene, ethane, carbon dioxide, and carbon monoxide, wherein the process gas has less than 50 ppmv of acetic acid (e.g., and less than 5 mol % of water vapor).

The cooling of the effluent downstream of the feed heat exchanger, thereby condensing water in the effluent, may include cooling the effluent in a quench vessel. The method may include separating in the quench vessel the effluent into gas and raw acetic acid, wherein the gas includes ethylene, water, acetic acid, ethane, carbon dioxide, and carbon monoxide, and wherein the raw acetic acid includes acetic acid and water. The method may include discharging the raw acetic acid from a bottom portion of the quench vessel to an acetic acid unit having an extractor column that is a liquid-liquid extraction column and processing the raw acetic acid in the acetic acid unit to give acetic acid product. The method may include discharging gas overhead from the quench vessel (as a quench tower or an acetic acid scrubber having a quench section), wherein the gas includes ethylene, water, acetic acid, ethane, carbon dioxide, and carbon monoxide, and removing water and acetic acid from the gas to give the process gas including ethylene, ethane, carbon dioxide, and carbon monoxide, wherein the process gas has less than 50 ppmv of acetic acid and less than 5 mol % of water vapor.

Yet another embodiment is a method of producing ethylene, including dehydrogenating ethane to ethylene via an ODH catalyst in presence of oxygen in an ODH reactor, thereby forming acetic acid in the ODH reactor, and discharging an effluent including ethylene, acetic acid, water, carbon monoxide, carbon dioxide, and unreacted ethane from the ODH reactor through a steam-generation heat exchanger to generate steam and through a feed heat exchanger to heat a feed including ethane for the ODH reactor. The method includes separating the effluent in a vessel into gas and raw acetic acid, wherein the gas includes ethylene, water, acetic acid, ethane, carbon dioxide, and carbon monoxide, and wherein the raw acetic acid includes acetic acid and water. The method includes removing acetic acid and water from the gas to give process gas including ethylene, ethane, carbon dioxide, and carbon monoxide and forwarding the process gas to a process gas compressor for further processing to give ethylene product, wherein the process gas includes less than 50 ppmv of acetic acid and less than 5 mol % of water vapor. The method includes discharging the raw acetic acid from a bottom portion of the vessel to an acetic acid unit (having an extractor column) to recover acetic acid product from the raw acetic acid.

The method may include discharging the gas overhead from the vessel, wherein the vessel is a flash drum or a quench tower. In other implementations, the removing the residual acetic acid and water from the gas occurs in the vessel, where the vessel in an acetic acid scrubber having a quenching section.

The method may include cooling the effluent with at least one of a cooler heat exchanger or an air cooler, wherein the cooler heat exchanger utilizes cooling water as a cooling medium, wherein the air cooler is a fan heat exchanger that utilizes air as a cooling medium.

Yet another embodiment is an ethylene production system including an ODH reactor having an ODH catalyst to dehydrogenate ethane to ethylene and generate acetic acid, a steam-generation heat exchanger to receive an effluent from the ODH reactor to generate steam with heat from the effluent, a feed heat exchanger to receive the effluent from the steam-generation heat exchanger to heat a feed including at least ethane for the ODH reactor with the effluent, and a vessel to separate the effluent into gas and raw acetic acid, wherein the gas includes ethylene, water, acetic acid, ethane, carbon dioxide, and carbon monoxide, and wherein the raw acetic acid includes acetic acid and water. The ethylene production system includes an acetic acid unit to process the raw acetic acid to give acetic acid product, wherein the acetic acid unit includes an extractor column that is a liquid-liquid extraction column.

The ethylene production system may include an acetic acid scrubber to remove acetic acid and water from the gas to give a process gas including ethylene, ethane, carbon dioxide, and carbon monoxide, wherein the process gas has less than 50 ppmv of acetic acid and less than 5 mol % of water vapor, and wherein the vessel is a flash drum or a quench tower. The acetic acid scrubber may have a quenching section. The ethylene production system may include a process gas compressor to receive the process gas for further processing to give ethylene product. The ethylene production system may include an ethane saturator tower to receive at least a portion of a bottom streams from the acetic acid scrubber as recycle water to saturate ethane with water for the feed including ethane.

The ethylene production system may include a cross-exchanger to receive at least a portion of a bottom streams from the acetic acid scrubber as recycle water to heat the recycle water, wherein the recycle water for an ethane saturator tower. The ethylene production system may include a cross-exchanger to heat a mixture with effluent downstream of the feed heat exchanger, wherein the mixture includes recycle water added to ethane gas for providing the feed including ethane, and wherein the recycle water includes at least a portion of a bottom streams from the acetic acid scrubber. The ethylene production system may include a cross-exchanger to heat a mixture with effluent downstream of the feed heat exchanger, wherein the mixture includes recycle water added to oxygen gas for providing the feed including ethane, and wherein the recycle water includes at least a portion of a bottom streams from the acetic acid scrubber. As mentioned, the system may have a vessel to separate the effluent into gas and raw acetic acid. The vessel may be an acetic acid scrubber to separate acetic acid and water from the gas to give process gas including ethylene, ethane, carbon dioxide, and carbon monoxide, and wherein the acetic acid scrubber has a quenching section.

The ethylene production system may include a heat exchanger to cool the effluent downstream of the feed heat exchanger, wherein the vessel is a flash drum. The heat exchanger may be a cooler heat exchanger that utilizes cooling water as a cooling medium, or the heat exchanger may be an air cooler including a fan heat exchanger that utilizes air as a cooling medium.

The ethylene production system may include a cross-exchanger to heat extract discharged from the extract column with the effluent, wherein the extract includes acetic acid and solvent. The acetic acid unit may have a solvent recovery column to receive the extract, and wherein the solvent recovery column is a distillation column. The ethylene production system may have a cross-exchanger to receive quench water discharged from a quench vessel to heat extract discharged from the extractor column, wherein the extract includes acetic acid, solvent, and water, and wherein the extract includes more solvent than water.

Yet another embodiment is a method of producing ethylene, including dehydrogenating ethane to ethylene via an ODH catalyst in an ODH reactor, and discharging an effluent from the ODH reactor, the effluent including ethylene, water, acetic acid, carbon dioxide, carbon monoxide, and unreacted ethane. The method includes condensing acetic acid and water in the effluent to separate the effluent into raw acetic acid and gas, the raw acetic acid including the condensed acetic acid and the condensed water, wherein the gas includes ethylene, carbon dioxide, carbon monoxide, and unreacted ethane. The method includes processing the raw acetic acid to give acetic acid product, and processing the gas to give process gas including ethylene product. The method includes recovering water from the effluent as recycle water, adding the recycle water to feed including ethane to the ODH reactor, heating the feed with the effluent, and adding oxygen to the feed. The method may include providing heat from the effluent to processing the raw acetic acid.

The processing of the gas to give the process gas may provide heat to processing the raw acetic acid. In implementations, the processing the gas heats ethane provided for the feed including the ethane. The method may include heating the recycle water with the effluent, and thereby cooling the effluent (and thus contribute to the cooling load to condense water and acetic acid in the effluent). In implementations, the processing the gas to give the process gas heats the recycle water. The method may include heating the feed including the ethane and recycle water with the effluent, and thereby cooling the effluent (and thus contribute to the cooling load to condense water and acetic acid in the effluent). The method may include heating the ethane provided for the feed with the effluent. The method may include heating the oxygen with the effluent.

The processing of the raw acetic acid may include the recovering water as recycle water. In other words, the processing of the raw acetic acid may provide for the action of recovering water from the effluent as recycle water. Moreover, the processing of the raw acetic acid to give acetic acid product and the processing the gas to give the process gas in combination may provide for the recovering the water from the effluent as recycle water.

A number of implementations have been described. Nevertheless, it will be understood that various modifications may be made without departing from the spirit and scope of the disclosure.

### INDUSTRIAL APPLICABILITY

The present disclosure relates to methods and systems for production of ethylene by oxidative dehydrogenation.

1. A method of producing ethylene, comprising:
dehydrogenating ethane to ethylene via an oxidative dehydrogenation (ODH) catalyst in presence of oxygen in an ODH reactor, thereby forming acetic acid in the ODH reactor;
discharging an effluent comprising ethylene, acetic acid, and water from the ODH reactor through a steam-generation heat exchanger to generate steam with heat from the effluent, thereby cooling the effluent;
flowing the effluent from the steam-generation heat exchanger through a feed heat exchanger to heat a feed comprising ethane for the ODH reactor with the effluent, thereby cooling the effluent;
recovering acetic acid from the effluent as acetic acid product; and
forwarding a process gas comprising ethylene from the effluent for further processing to give ethylene product.

2. (canceled)

3. The method of claim 1, comprising further cooling the effluent downstream of the feed heat exchanger, thereby condensing water in the effluent, wherein further cooling the effluent downstream of the feed heat exchanger comprises cooling the effluent with at least one of a cooler heat exchanger or an air cooler, wherein the cooler heat exchanger utilizes cooling water as a cooling medium, and wherein the air cooler comprises a fan heat exchanger that utilizes air as a cooling medium.

4. The method of claim 1, comprising further cooling the effluent downstream of the feed heat exchanger, thereby condensing water in the effluent, wherein further cooling the effluent downstream of the feed heat exchanger comprises cooling the effluent in a quench tower or in an acid scrubber having a quenching section.

5. The method of claim 1, comprising further cooling the effluent downstream of the feed heat exchanger, thereby condensing water in the effluent, comprising separating the effluent as further cooled and having the water as condensed into raw acetic acid and gas, wherein the gas comprises ethylene, water, acetic acid, ethane, carbon dioxide, and carbon monoxide, and wherein the raw acetic acid comprises acetic acid and water.

6. The method of claim 5, wherein further cooling the effluent downstream of the feed heat exchanger and separating the effluent as further cooled into gas and raw acetic acid comprises processing the effluent in a quench tower or in an acetic acid scrubber having a quenching section, and wherein the method comprises discharging the raw acetic acid from a bottom portion of the quench tower or from a bottom portion of the acetic acid scrubber having the quenching section.

7. The method of claim 5, wherein further cooling the effluent downstream of the feed heat exchanger comprises cooling the effluent in a heat exchanger, wherein separating the effluent comprises separating the effluent in a flash drum into the gas and the raw acetic acid, and wherein the method comprises discharging the gas overhead from the flash drum and discharging the raw acetic acid from a bottom portion of the flash drum.

8. The method of claim 5, comprising removing water and acetic acid from the gas to give the process gas comprising the ethylene, ethane, carbon dioxide, and carbon monoxide, wherein the process gas comprises less than 50 parts per million volume (ppmv) of acetic acid and less than 5 mole percent of water vapor.

9. The method of claim 8, comprising providing water and acetic acid removed from the gas as recycle water for saturating the feed comprising ethane with water.

10. The method of claim 9, comprising heating at least a portion of the recycle water in a cross-exchanger with the effluent as a heating medium.

11. The method of claim 9, comprising:
combining at least a portion of the recycle water with oxygen gas to give a mixture;
heating the mixture in a cross-exchanger with effluent as a heating medium; and
adding the mixture as heated to the feed comprising the ethane.

12. (canceled)

13. (canceled)

14. The method of claim 5, comprising providing the raw acetic acid to an acetic acid unit comprising an extractor column that is a liquid-liquid extraction column, wherein recovering acetic acid from the effluent as acetic acid product comprises processing the raw acetic acid in the acetic acid unit, wherein processing the raw acetic acid in the acetic acid unit comprises:
providing the raw acetic acid and solvent to the extractor column;
discharging extract overhead from the extractor column, wherein the extract comprises primarily acetic acid and further comprises the solvent and water, wherein the extract comprises more of the solvent than water; and
heating the extract in a cross-exchanger with a heating medium, wherein the heating medium comprises the effluent downstream of the feed heat exchanger, or wherein the heating medium comprises quench water.

15. The method of claim 4, comprising discharging the acetic acid product comprising at least 99 weight percent (wt %) acetic acid from the acetic acid unit, wherein the raw acetic acid comprises a concentration of acetic acid in a range of 0.3 wt % to 45 wt %, and wherein the acetic acid unit comprises a solvent recovery column that is a distillation column.

16. A method of producing ethylene, comprising:
dehydrogenating ethane to ethylene via an oxidative dehydrogenation (ODH) catalyst in presence of oxygen in an ODH reactor, thereby forming acetic acid in the ODH reactor;
discharging an effluent comprising ethylene, acetic acid, water, carbon monoxide, and carbon dioxide from the ODH reactor through a steam-generation heat exchanger to generate steam, wherein the steam-generation heat exchanger transfers heat from the effluent to water to generate the steam, thereby cooling the effluent;
flowing the effluent from the steam-generation heat exchanger through a feed heat exchanger to heat a feed for the ODH reactor with the effluent, wherein the feed heat exchanger transfers heat from the effluent to the feed, thereby cooling the effluent;
cooling the effluent downstream of the feed heat exchanger, thereby condensing water in the effluent; and
forwarding process gas comprising ethylene from the effluent to a process gas compressor for further processing to give ethylene product.

17. (canceled)

18. The method of claim 16, comprising:
providing the effluent having the condensed water to a flash drum; and
separating in the flash drum the effluent into gas and raw acetic acid, wherein the gas comprises ethylene, water, acetic acid, ethane, carbon dioxide, and carbon monoxide, and wherein the raw acetic acid comprises acetic acid and water.

19. The method of claim 18, comprising:
discharging the raw acetic acid from a bottom portion of the flash drum to an acetic acid unit comprising an extractor column that is a liquid-liquid extraction column and a solvent recovery column that is a distillation column; and
processing the raw acetic acid in the acetic acid unit to give acetic acid product.

20. The method of claim 18, comprising:
discharging the gas overhead from the flash drum; and
removing water and acetic acid from the gas to give the process gas comprising the ethylene, ethane, carbon dioxide, and carbon monoxide, wherein the process gas comprises less than 50 parts per million volume (ppmv) of acetic acid and less than 5 mole percent of water vapor.

21. (canceled)

22. (canceled)

23. The method of claim 16, comprising:
separating in the quench vessel the effluent into gas and raw acetic acid, wherein the gas comprises ethylene, water, acetic acid, ethane, carbon dioxide, and carbon monoxide, and wherein the raw acetic acid comprises acetic acid and water;
discharging the raw acetic acid from a bottom portion of the quench vessel to an acetic acid unit comprising an extractor column that is a liquid-liquid extraction column; and
processing the raw acetic acid in the acetic acid unit to give acetic acid product

24. The method of claim 16, comprising:
discharging gas overhead from the quench vessel, wherein the gas comprises ethylene, water, acetic acid, ethane, carbon dioxide, and carbon monoxide, and wherein the quench vessel comprises a quench tower; and
removing water and acetic acid from the gas to give the process gas comprising ethylene, ethane, carbon dioxide, and carbon monoxide, wherein the process gas comprises less than 50 parts per million volume (ppmv) of acetic acid and less than 5 mole percent of water vapor.

25. The method of claim 16, comprising removing acetic acid and water from the gas to give the process gas and discharging the process gas overhead from the quench vessel, wherein the quench vessel comprises an acetic acid scrubber having a quenching section.

26. (canceled)

27. (canceled)

28. (canceled)

29. (canceled)

30. An ethylene production system comprising:
an oxidative dehydrogenation (ODH) reactor comprising an ODH catalyst to dehydrogenate ethane to ethylene and generate acetic acid;
a steam-generation heat exchanger to receive an effluent from the ODH reactor to generate steam with heat from the effluent;
a feed heat exchanger to receive the effluent from the steam-generation heat exchanger to heat a feed comprising ethane for the ODH reactor with the effluent;
a vessel to separate the effluent into gas and raw acetic acid, wherein the gas comprises ethylene, water, acetic acid, ethane, carbon dioxide, and carbon monoxide, and wherein the raw acetic acid comprises acetic acid and water; and
an acetic acid unit to process the raw acetic acid to give acetic acid product, wherein the acetic acid unit comprises an extractor column that is a liquid-liquid extraction column.

31. The system of claim 30, comprising an acetic acid scrubber to remove acetic acid and water from the gas to give a process gas comprising ethylene, ethane, carbon dioxide, and carbon monoxide, wherein the process gas comprises less than 50 parts per million volume (ppmv) of acetic acid and less than 5 mole percent of water vapor, and wherein the vessel comprises a flash drum or a quench tower.

32. (canceled)

33. (canceled)

34. The system of claim 31, comprising an ethane saturator tower to receive at least a portion of a bottom streams from the acetic acid scrubber as recycle water to saturate ethane with water for the feed comprising ethane.

35. The system of claim 31, comprising a cross-exchanger to receive at least a portion of a bottom streams from the acetic acid scrubber as recycle water to heat the recycle water, wherein the recycle water for an ethane saturator tower.

36. The system of claim 31, comprising a cross-exchanger to heat a mixture with effluent downstream of the feed heat exchanger, wherein the mixture comprises recycle water added to ethane gas for providing the feed comprising ethane, and wherein the recycle water comprises at least a portion of a bottom streams from the acetic acid scrubber.

37. The system of claim 31, comprising a cross-exchanger to heat a mixture with effluent downstream of the feed heat exchanger, wherein the mixture comprises recycle water added to oxygen gas for providing the feed comprising ethane, and wherein the recycle water comprises at least a portion of a bottom streams from the acetic acid scrubber.

38. (canceled)

39. (canceled)

40. (canceled)

41. The system of claim 30, comprising a heat exchanger to cool the effluent downstream of the feed heat exchanger, wherein the vessel comprises a flash drum, wherein the heat exchanger comprises (a) a cooler heat exchanger that utilizes cooling water as a cooling medium or (b) an air cooler comprising a fan heat exchanger that utilizes air as a cooling medium.

42. The system of claim 30, comprising a cross-exchanger to heat extract discharged from the extractor column with the effluent, wherein the extract comprises acetic acid and solvent.

43. (canceled)

44. The system of claim 30, comprising a cross-exchanger to receive quench water discharged from a quench vessel to heat extract discharged from the extractor column, wherein the extract comprises acetic acid, solvent, and water, and wherein the extract comprises more solvent than water.

45. (canceled)

46. (canceled)

47. (canceled)

48. (canceled)

49. (canceled)

50. (canceled)

51. (canceled)

52. (canceled)

53. (canceled)

54. (canceled)

55. (canceled)
